(19) United States
(12) Patent Application Publication
(10) Pub. No.: US 2021/0395736 A1
(43) Pub. Date: Dec. 23, 2021

(54) RASOPATHY TREATMENT

(71) Applicant: The Johns Hopkins University, Baltimore, MD (US)

(72) Inventors: Renyuan Bai, Joppa, MD (US); Verena Staedtke, Baltimore, MD (US)

(21) Appl. No.: 17/289,620

(22) PCT Filed: Oct. 29, 2019
(86) PCT No.: PCT/US2019/058447

§ 371 Date: Apr. 28, 2021

Related U.S. Application Data
(60) Provisional application No. 62/751,968, filed on Oct. 29, 2018.

(51) Int. Cl. C12N 15/113; C12N 15/86; C07K 14/47

(52) U.S. Cl. CPC C12N 15/113; C07K 14/47; C12N 15/86

(57) ABSTRACT

Provided herein is technology relating to treatment of Ras-associated diseases and particularly, but not exclusively, to compositions, methods, systems, and kits for decreasing Ras activity using a neurofibromin 1 GTPase-activating protein-related domain gene therapy construct.

This application claims priority to U.S. provisional patent application Ser. No. 62/751,968, filed Oct. 29, 2018, which is incorporated herein by reference in its entirety.

### STATEMENT REGARDING FEDERALLY SPONSORED RESEARCH OR DEVELOPMENT

This invention was made with government support under CA230179 awarded by the National Institutes of Health and W81XWH1810236 awarded by the Department of Defense. The government has certain rights in the invention.

### FIELD

Provided herein is technology relating to treatment of Ras-associated diseases and particularly, but not exclusively, to compositions, methods, systems, and kits for decreasing Ras activity using a neurofibromin 1 GTPase-activating protein-related domain gene therapy construct.

### BACKGROUND

The Rasopathy neurofibromatosis 1 (Online Mendelian Inheritance in Man (OMIM) #162200; also known as von Recklinghausen disease) is an autosomal dominant hereditary cancer syndrome that affects approximately 1:3,000 individuals [1, 2]. Neurofibromatosis 1 (NF-1) is caused by DNA alterations including point mutations, deletions, insertions, microdeletions, and/or splicing mutations of the neurofibromin 1 (NF1) tumor suppressor gene at 17q11.2, which encodes the GTPase-activating protein (GAP) NF1. NF1 catalyzes the inactivation of Ras by accelerating Ras guanosine triphosphatase activity (e.g., GTP hydrolysis to GDP) [3, 4]. In affected individuals, truncation or loss of NF1 results in aberrantly activated Ras with subsequent activation of the RAF-MEK-ERK cascade. Consequently, aberrant Ras activation (e.g., hyperactivation) causes development of multiple benign tumors (e.g., neurofibromas) and malignancies (e.g., malignant peripheral nerve sheath tumors (MPNSTs)). Although MPNSTs occur only in a fraction of NF-1 individuals, this cancer is the leading cause of death in this patient population: despite most aggressive interventions there is only a low chance of long-term survival and approximately 60-80% of patients develop recurrences within 5 years after initial diagnosis, thus reflecting the need for new and more effective therapeutics for these cancers.

### SUMMARY

Accordingly, provided herein is technology relating to treatment of Ras-associated diseases and particularly, but not exclusively, to compositions, methods, systems, and kits for decreasing Ras activity using a neurofibromin 1 GTPase-activating protein-related domain gene therapy construct.

Gene therapy has a potential for application to diseases that were previously difficult to treat or untreatable. Although the strategy of replacing a malfunctioning gene is conceptually simple, in practice gene therapy is complicated by several technical difficulties, e.g., biological barriers associated with use of the technology in particular cells and tissues and immunological barriers related to patient recognition of therapies as non-self. One particular issue affecting use of gene therapy technologies relates to maximizing general and proficient expression of a therapeutic molecule in a sufficient number of gene-corrected cells to treat the condition without disrupting essential regulatory mechanisms and without increasing the risks of side effects from genomic manipulation and off-target effects [14, 15]. These challenges have been intensively researched over the last years, leading to safer, more effective therapies and unprecedented treatment successes for some monogenic disorders [13-15].

While AAVs are known in the art for gene therapy uses, one challenge facing therapies using AAVs is the poor efficacy in delivery, to which many efforts have been directed, e.g., by modifying and optimizing the AAV cap gene [30]. For example, a recombinant AAV-DJ was constructed to improve targeting and reduce anti-AAV immunity. This vector was constructed by DNA shuffling and immuno-selection from cap genes of 8 natural serotypes experimental tests indicated that it outperformed all of the parental AAV vectors [25]. Further engineering of AAV-DJ resulted in improved delivery in various mice tissues [25, 31].

During the development of embodiments of the technology provided herein, experiments were conducted in which the transduction efficacies of several recombinant AAV vectors were evaluated in MPNST cells and human Schwann cells. The panel of vectors tested comprise vectors having distinct capsids from 12 natural serotypes of human and monkey origins and the synthetic AAV-DJ vectors. The data indicated that AAV1, AAV2, AAV3B, AAV-DJ, AAV6 transduced HSC and NF90-8 particularly efficiently. Accordingly, these vectors are appropriate for testing and use in vivo and, in some embodiments, provide templates for additional engineering to improve targeting in MPNST and HSC in vitro and further in vivo.

Furthermore, the technology comprises use of the GRD domain of NF1. In some embodiments, the NF1-GRD is delivered into a cell (e.g., a tissue, an organism, a patient) by an AAV vector as described herein. NF1 is a large protein with various domains, e.g., including a cysteine serine-rich domain (CSRD), a tubulin-binding domain (TBD), a GRD, a Sec14, a PH-like domain, and a FAK-binding region [33]. A GRD comprising 333 amino acids (NF1-333, corresponding to amino acids 1198-1530 of NF1) has been studied previously. This GRD comprises a central minimal GAP domain of 230 amino acids (amino acids 1248-1477 of NF1) and flanking extra regions that mediate important GAP-related functions and interactions [33-35]. In physiological conditions, the NF1 protein is recruited to the plasma membrane by interacting with Spred1. In particular, the Spred1 EVH1 domain associates with the N-terminal region (amino acids 1202-1217 of NF1) and C-terminal region (amino acids 1511-1530 of NF1) of GRD [36-38].

During the development of embodiments of the technology provided herein, experiments were conducted using a GRD polypeptide comprising amino acids 1172-1538 of NF1 and a double HA tag that is provided for the convenience of protein detection [39]. While embodiments of the technology described herein comprise use of an HA tag for detection (e.g., during experiments conducted during the development of the technology provided herein, e.g., for detection of engineered GRD expression via antibody staining in immunoblots, enzyme-linked immunosorbent assay (ELISA), and/or by immunofluorescence), the technology is not limited to polypeptides comprising a tag. Accordingly, the therapeutic uses of the technology provided herein are contemplated to comprise use of a GRD polypeptide that may or may not comprise a tag. In some embodiments, expression of engineered GRD protein is monitored in a human during therapeutic use without use of a tag. In some embodiments, a tag finds use in monitoring expression of engineered GRD protein (e.g., during experimental use and/or in a human during therapeutic use). In embodiments comprising use of a tag, the technology is not limited to use of an HA tag. Accordingly, the technology comprises use of any tag for detection, isolation, etc. (e.g., 6× His, FLAG, V5, myc, fluorescent, and other tags known in the art).

Data collected during the experiments indicated that this GRD construct presented a diffused subcellular distribution in MPNST cells. During post-translational modification of Ras proteins, the cysteine residue of “CAAX” is prenylated (e.g., farnesylated) and subsequently the “AAX” part at the C-terminus is cleaved away. The prenylated cysteine is further methylesterified, creating a hydrophobic tail at the C-terminus, which promotes Ras processing but does not provide efficient plasma membrane targeting. Additional palmitoylation of the cysteine residues and/or the polybasic sequence in the Ras HVR (FIG. GA) are required for localization in the plasma membrane [43]. Accordingly, GRD was targeted to the plasma membrane and thus to Ras proteins by fusing GRD to a sequence from a CAAX motif-containing partial or complete Ras HVR sequence, e.g., a sequence from a Ras HVR (e.g., a partial or complete Ras HVR sequence (e.g., comprising a CAAX motif)). In some embodiments, a number of amino acids from the C-terminus of a Ras HVR are used. For example, in some embodiments, the 10 C-terminal amino acids are used (abbreviated C10); in some embodiments, the 24 C-terminal amino acids are used (abbreviated C24). In some embodiments, the 4, 5, 6, 7, 8, 9, 10, 11, 12, 13, 14, 15, 16, 17, 18, 19, 20, 21, 22, 23, or 24 C-terminal amino acids are used (abbreviated C4, C5, C6, C7, C8, C9, C10, C11, C12, C13, C14, C15, C16, C17, C18, C19, C20, C21, C22, C23, or C24, respectively).

In some embodiments, GRD is fused to a N-Ras HVR sequence (C24), K-Ras4A HVR sequences, or KRas4B HVR sequences (C21 and C24) to improve targeting to membrane-associated Ras proteins. These constructs (GRD-C10, GRD-HRas-C24, GRD-NRas-C24, GRD-KRas4A-C22 and GRD-KRas4A-C24, GRD-KRas4B-C21 and GRD-KRas-4B-C24) markedly improved the potency of GRD in suppressing pErk1/2 (represented by data with GRD-C10 and GRD-KRas4B-C24) and the growth of NF1-related MPNST and NF-1 plexiform cells with a remarkable specificity in contrast to NF1-unrelated MPNST cells and HSC (represented by data with GRD-C10). Thus, given its potency and specificity, embodiments of the technology provide compositions comprising GRD fusion proteins with CAAX motif-containing partial or complete Ras HVR sequences (GRD-C10, GRD-HRas-C24, GRD-NRas-C24, GRD-KRas4A-C22 and GRD-KRas4A-C24, GRD-KRas4B-C21 and GRD-KRas-4B-C24), methods using GRD fusion proteins with CAAX-containing Ras HVR sequences, and related kits, systems, and uses for gene replacement therapy in MPNST, NF-1 plexiform and NF1-haploid individuals.

Accordingly, embodiments of the technology provide a recombinant polypeptide comprising a neurofibromin 1 GTPase-activating protein-related domain (NF1-GRD) and a membrane-targeting amino acid sequence. The technology is not limited in the membrane-targeting amino acid sequence and includes any membrane-targeting amino acid sequence (e.g., peptide) known in the art. For example, in some embodiments, the membrane-targeting amino acid sequence comprises CAAX motif-containing partial or complete Ras HVR sequence, e.g., a sequence from a Ras HVR (e.g., a partial or complete Ras HVR sequence (e.g., comprising a CAAX motif)). In some embodiments, the membrane-targeting amino acid sequence comprises the amino acid sequence GCMSCKCVLS (SEQ ID NO: 3). In some embodiments, the membrane-targeting amino acid sequence comprises a sequence from SEQ ID NOs: 10, 11, 12, or 13 (e.g., 1 to 24 (e.g., 1, 2, 3, 4, 5, 6, 7, 8, 9, 10, 11, 12, 13, 14, 15, 16, 17, 18, 19, 20, 21, 22, 23, or 24) C-terminal amino acids from SEQ ID NOs: 10, 11, 12, or 13). In some embodiments, the membrane-targeting amino acid sequence comprises a sequence from SEQ ID NOs: 14, 15, 16, 17, 18, 19, and/or 20.

In some embodiments, the NF1-GRD amino acid sequence comprises an amino acid sequence provided by SEQ ID NO: 2. In some embodiments, the NF1-GRD amino acid sequence comprises an amino acid sequence from approximately amino acid 1150 to approximately amino acid 1600 of the amino acid sequence provided by SEQ ID NO: 2. In some embodiments, the recombinant polypeptide comprises the amino acid sequence from approximately amino acid 1150 to approximately amino acid 1600 of the NF1 protein amino acid sequence as provided by SEQ ID NO: 2 and/or NCBI Accession NP_000258.1. In some embodiments, the recombinant polypeptide comprises the amino acid sequence from approximately amino acid 1150 (e.g., approximately amino acid 1150 to approximately amino acid 1200 (e.g., amino acid 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200)) to approximately amino acid 1550 (e.g., approximately amino acid 1500 to approximately amino acid 1550 (e.g., amino acid 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, or 1550)) of the NF1 protein provided by SEQ ID NO: 2 and/or NCBI Accession NP 000258.1.

In some embodiments, the membrane-targeting amino acid sequence is joined in-frame to the C-terminus of said NF1-GRD.

The technology further provides embodiments of a recombinant nucleic acid comprising a nucleotide sequence encoding a NF1-GRD and a membrane-targeting amino acid sequence. In some embodiments, the nucleotide sequence encoding said NF1-GRD comprises a nucleotide sequence provided by SEQ ID NO: 1. In some embodiments, the nucleic acid comprises a nucleotide sequence provided by SEQ ID NO: 1 and/or NCBI Accession NM_000267.3. In some embodiments, the nucleotide sequence encoding the NF1-GRD comprises a nucleotide sequence from approximately nucleotide 3448 to approximately nucleotide 4650 of the nucleotide sequence provided by SEQ ID NO: 1. In some embodiments, the nucleic acid comprises a nucleotide sequence provided by the sequence beginning within the range of approximately nucleotide 3448 to 3600 (e.g., 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, or 3600) and ending within the range of approximately nucleotide 4498 to 4650 (e.g., 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554, 4555, 4556, 4557, 4558, 4559, 4560, 4561, 4562, 4563, 4564, 4565, 4566, 4567, 4568, 4569, 4570, 4571, 4572, 4573, 4574, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4601, 4602, 4603, 4604, 4605, 4606, 4607, 4608, 4609, 4610, 4611, 4612, 4613, 4614, 4615, 4616, 4617, 4618, 4619, 4620, 4621, 4622, 4623, 4624, 4625, 4626, 4627, 4628, 4629, 4630, 4631, 4632, 4633, 4634, 4635, 4636, 4637, 4638, 4639, 4640, 4641, 4642, 4643, 4644, 4645, 4646, 4647, 4648, 4649, or 4650) of SEQ ID NO: 1.

In some embodiments, the nucleic acid comprises a nucleotide sequence encoding a CAAX motif-containing partial or complete Ras HVR sequence, e.g., a sequence from a

Ras HVR (e.g., a partial or complete Ras HVR sequence (e.g., a sequence comprising a CAAX motif)). In some embodiments, the nucleic acid comprises a nucleotide sequence encoding the amino acid sequence GCMSCKCVLS (SEQ ID NO: 3). In some embodiments, the nucleotide sequence encodes said membrane-targeting amino acid sequence joined in-frame to the C-terminus of said NF1-GRD.

Additional embodiments provide a vector comprising a nucleic acid comprising a recombinant nucleotide sequence encoding a NF1-GRD and a membrane-targeting amino acid sequence. In some embodiments, the vector is a gene-delivery vector. In some embodiments, the vector is an adeno-associated virus (AAV) vector. In some embodiments, the vector is an AAV1, AAV2, AAV3B, AAV6, or AAV-DJ AAV vector.

In some embodiments, the technology provides a gene-delivery vehicle. For instance, in some embodiments, the technology provides a virus comprising a nucleic acid comprising a recombinant nucleotide sequence encoding a NF1-GRD and a membrane-targeting amino acid sequence.

In some embodiments, the technology relates to cells, tissues, and organisms. For example, in some embodiments, the technology provides a cell comprising a recombinant nucleic acid comprising a nucleotide sequence encoding a NF1-GRD and a membrane-targeting amino acid sequence. In some embodiments, the cell comprises a recombinant nucleic acid and the recombinant nucleic acid is integrated into the genome. In some embodiments, the cell comprises a recombinant nucleic acid and the recombinant nucleic acid is present in a vector. Some embodiments provide a tissue comprising a cell comprising a recombinant nucleic acid comprising a nucleotide sequence encoding a NF1-GRD and a membrane-targeting amino acid sequence. Some embodiments provide an organism comprising a recombinant nucleic acid comprising a nucleotide sequence encoding a NF1-GRD and a membrane-targeting amino acid sequence.

In related embodiments, the technology provides a cell expressing a recombinant nucleic acid comprising a nucleotide sequence encoding a NF1-GRD and a membrane-targeting amino acid sequence. In some embodiments, the technology provides a cell expressing a recombinant polypeptide comprising a NF1-GRD and a membrane-targeting amino acid sequence. In some embodiments, the cell comprises aberrant Ras activity. In some embodiments, the cell comprises a mutation in a NF1 gene. In some embodiments, the cell is a cancer or tumor cell. In some embodiments, the cell is a Schwann cell. In some embodiments, the cell is a MPNST cell.

In some embodiments, a subject comprises a cell or tissue in need of treatment according to the technology provided herein. In some embodiments, a subject having a Rasopathy comprises a cell expressing a recombinant nucleic acid comprising a nucleotide sequence encoding a NF1-GRD and a membrane-targeting amino acid sequence and/or a cell expressing a recombinant polypeptide comprising a NF1-GRD and a membrane-targeting amino acid sequence. In some embodiments, a subject having a neurofibroma or neurofibromatosis 1 comprises a cell expressing a recombinant nucleic acid comprising a nucleotide sequence encoding a NF1-GRD and a membrane-targeting amino acid sequence and/or a cell expressing a recombinant polypeptide comprising a NF1-GRD and a membrane-targeting amino acid sequence. In some embodiments, a subject having a MPNST comprises a cell expressing a recombinant nucleic acid comprising a nucleotide sequence encoding a NF1-GRD and a membrane-targeting amino acid sequence and/or a cell expressing a recombinant polypeptide comprising a NF1-GRD and a membrane-targeting amino acid sequence.

Some embodiments relate to pharmaceutical compositions. For example, in some embodiments, the technology provided herein relates to a pharmaceutical composition comprising a recombinant nucleic acid comprising a nucleotide sequence encoding a NF1-GRD and a membrane-targeting amino acid sequence. In some embodiments, the pharmaceutical composition comprises a gene-delivery vector comprising said recombinant nucleic acid. In some embodiments, the pharmaceutical composition provides the recombinant nucleic acid in a therapeutically effective dose. In some embodiments, the pharmaceutical composition is formulated for administration to a subject. In some embodiments, the pharmaceutical composition is formulated for administration to a tissue.

In some embodiments, the technology provides a kit comprising a composition described herein. For example, in some embodiments, the technology provides a kit comprising a pharmaceutical composition comprising a recombinant nucleic acid comprising a nucleotide sequence encoding a NF1-GRD and a membrane-targeting amino acid sequence and a means for administration.

The technology further encompasses embodiments of methods. For example, in some embodiments, the technology provides a method of treating a subject having a Rasopathy. In some embodiments, the method comprises administering a recombinant nucleic acid comprising a nucleotide sequence encoding a NF1-GRD and a membrane-targeting amino acid sequence to a subject. In some embodiments, the methods reduce Ras activity in the subject. In some embodiments, methods comprise administering a recombinant nucleic acid comprising a nucleotide sequence encoding a NF1-GRD and a membrane-targeting amino acid sequence to a subject who has or is at risk of developing a neurofibroma, a neurofibromatosis, or a MPNST. In some embodiments, the subject has a mutation in the NF1 gene. In some embodiments, the methods ameliorate one or more symptoms of a disease. For example, in some embodiments, the administration of the recombinant nucleic acid comprising a nucleotide sequence encoding a NF1-GRD and a membrane-targeting amino acid sequence results in a reduced tumor load in the subject. In some embodiments, the subject is a human. Methods comprise administering the recombinant nucleic acid comprising a nucleotide sequence encoding a NF1-GRD and a membrane-targeting amino acid sequence in combination with an additional therapeutic agent or medical intervention known in the art (e.g., surgery, chemotherapy, radiotherapy, immunotherapy, etc.).

In some embodiments, methods comprise identifying a subject in need of treatment according to the methods described herein, e.g., administering the recombinant nucleic acid comprising a nucleotide sequence encoding a NF1-GRD and a membrane-targeting amino acid sequence. In some embodiments, methods comprise monitoring a subject treated according to the methods described herein, e.g., by administering the recombinant nucleic acid comprising a nucleotide sequence encoding a NF1-GRD and a membrane-targeting amino acid sequence. In some embodiments, identifying and/or monitoring a subject comprises testing the subject for a symptom and/or marker of disease. For example, in some embodiments, methods further comprise testing a subject for a NF1 mutation. In some embodiments, methods further comprise testing the subject for aberrant Ras activity. In some embodiments, methods further comprise testing the subject for a cancer. In some embodiments, methods further comprise testing the subject for a neurofibroma or MPNST.

Some embodiments comprise acting based on the outcome of testing the subject. For example, in some embodiments, methods further comprise the step of administering a second dose of said recombinant nucleic acid after the testing step.

The technology finds use in various applications. For example, embodiments of the technology relate to use of a recombinant nucleic acid comprising a nucleotide sequence encoding a NF1-GRD and a membrane-targeting amino acid sequence to prepare a medicament. Some embodiments relate to use of a recombinant nucleic acid comprising a nucleotide sequence encoding a NF1-GRD and a membrane-targeting amino acid sequence to prepare a medicament for treating a subject having a Rasopathy, neurofibroma, neurofibromatosis type 1, and/or a MPNST disease. Some embodiments relate to use of a recombinant nucleic acid comprising a nucleotide sequence encoding a NF1-GRD and a membrane-targeting amino acid sequence to provide a transgenic organism. Some embodiments relate to use of a recombinant nucleic acid comprising a nucleotide sequence encoding a NF1-GRD and a membrane-targeting amino acid sequence to provide a gene therapy construct. Some embodiments relate to use of a recombinant nucleic acid comprising a nucleotide sequence encoding a NF1-GRD and a membrane-targeting amino acid sequence in research. Some embodiments relate to use of a recombinant nucleic acid comprising a nucleotide sequence encoding a NF1-GRD and a membrane-targeting amino acid sequence in studying disease in a model system (e.g., a mammal (e.g., a mouse, rat, dog) and/or a cell culture system).

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1A is an image of a western blot showing NF1 expression in MPNST cells. Western blotting was performed with the lysates of NF1-related MPNST cells (NF90-8, sNF96.2, and ST88-14), NF1-unrelated MPNST STS26T, and human Schwann cells (HSC). Beta-actin was used as a loading control.

FIG. 1B is a series of images from western blots showing that overexpression of NF1-GRD reduced Ras activities in MPNST cells. ST88-14 and NF90-8 were transduced by EGFP or GRD-2HA lentivirus and cell lysates were subjected to pulldown assay with GST-Raf-RBD. Western blotting was performed with the indicated antibodies to Ras, beta-actin, or hemagglutinin. GTP_{Y}S and GDP provided the positive and negative controls, respectively.

FIG. 1C is a bar plot showing that NF1-GRD suppressed the growth of MPNST cells. MPNST cells were transduced with EGFP or GRD-2HA via lentivirus in 96-well plates and viable cells were measured after 72 hours. P values were evaluated by two-tailed t-test.

FIG. 2A is a bar plot showing the transduction efficacies of different AAV vectors in ST88-14 cells. ST88-14 cells plated in 96-well plates were transduced with the indicated AAVs at multiplicity of infection (MOI) 1000 or 5000. Green fluorescent signals were measured after 5 days. Statistical significance was evaluated by two-tailed t-test.

FIG. 2B is a series of fluorescence images of EGFP in ST88-14 cells transduced by selected AAVs. Nuclei were stained by NucBlue Live Cell Stain.

FIG. 2C is a bar plot showing the transduction efficacies of different AAV vectors in NF90-8 cells. NF90-8 cells plated in 96-well plates were transduced with the indicated AAVs at MOI 1000 or 5000. Green fluorescent signals were measured after 5 days.

FIG. 2D is a series of fluorescence images of EGFP in NF90-8 cells transduced by selected AAVs. Nuclei were stained by NucBlue Live Cell Stain.

FIG. 3A is a bar plot showing the transduction efficacies of different AAV vectors in sNF96.2 cells. sNF96.2 cells plated in 96-well plates were transduced with the indicated AAVs at MOI 1000 or 5000. Green fluorescent signals were measured after 5 days. Statistical significance was evaluated by two-tailed t-test.

FIG. 3B is a series of fluorescence images of EGFP in sNF96.2 cells transduced by selected AAVs. Nuclei were stained by NucBlue Live Cell Stain.

FIG. 3C is a fluorescence image showing green fluorescence staining of human spinal nerve Schwann cells with anti-S100 antibody.

FIG. 3D is a bar plot showing the transduction efficacies of different AAV vectors in human Schwann cells. HSC plated in 96-well plates were transduced with the indicated AAVs at MOI 1000 or 5000. Green fluorescent signals were measured after 5 days.

FIG. 3E is a series of fluorescence images of EGFP in HSC transduced by selected AAVs. Nuclei were stained by NucBlue Live Cell Stain.

FIG. 4A is an image of a western blot showing the transduction of GRD-2HA and 2HA-GRD-C10 in NF90-8 cells by AAV-DJ. NF90-8 cells were transduced at MOI 5000 by AAV-DJ carrying EGFP (control), GRD-2HA, or 2HA-GRD-C10. The anti-HA western blot showed the expression of GRD-2HA and 2HAGRD-C10, and the anti-pERK1/2 blot demonstrated the complete inhibition of ERK1/2 (p42/44) phosphorylation by the GRD-C10 construct. The anti-ERK1/2 blot showed the total levels of ERK1/2 in the lysates, whereas the anti-beta-actin blot was used as a control.

FIG. 4B is a series of anti-HA immunofluorescence images of NF90-8 cells transduced with GRD-2HA or 2HA-GRD-C10 by AAV-DJ. Nuclei were stained by DAPI.

FIG. 4C is a bar plot showing the inhibition of MPNST cells by GRD and GRD-C10 transduced by AAV-DJ. ST88-14 cells were plated in 96-well plates and incubated with AAV-DJ carrying EGFP, GRD-2HA, or 2HA-GRD-C10 constructs at MOI 5000. After 4 days, viable cells were measured. P values were evaluated by two-tailed t-test.

FIG. 4D is a bar plot showing the inhibition of MPNST cells by GRD and GRD-C10 transduced by AAV-DJ. NF90-8 cells were plated in 96-well plates and incubated with AAV-DJ carrying EGFP, GRD-2HA, or 2HA-GRD-C10 constructs at MOI 5000. After 4 days, viable cells were measured. P values were evaluated by two-tailed t-test.

FIG. 4E is a bar plot showing the inhibition of MPNST cells by GRD and GRD-C10 transduced by AAV-DJ. sNF96.2 cells were plated in 96-well plates and incubated with AAV-DJ carrying EGFP, GRD-2HA, or 2HA-GRD-C10 constructs at MOI 5000. After 4 days, viable cells were measured. P values were evaluated by two-tailed t-test.

FIG. 5A is a bar plot showing the transduction efficacies of different AAV vectors in STS26T cells. Cells plated in 96-well plates were transduced with indicated AAVs at MOI 5000. Green fluorescent signals were measured after 5 days. Statistical significance was evaluated by two-tailed t-test.

FIG. 5B is a series of fluorescence images of EGFP in STS26T cells transduced by indicated AAVs. Nuclei were stained by NucBlue Live Cell Stain.

FIG. 5C is a bar plot showing that STS26C cells, a spontaneous MPNST cell line with intact NF1 (FIG. 1A), were not affected by GRD and GRD-C10 transduced by AAV-DJ. STS26T MPNST cells and human Schwann cells were plated in 96 well plates and incubated with AAV-DJ carrying EGFP, GRD-2HA or 2HA-GRD-C10 constructs at MOI 5000. After 4 days, viable cells were measured. Statistical significance was evaluated by two-tailed t-test. NS: not significant, p>0.05.

FIG. 5D is a bar plot showing that growth of HSCs was not suppressed by GRD and was not suppressed by GRD-C10 at a significant level. Human Schwann cells were plated in 96-well plates and incubated with AAV-DJ carrying EGFP, GRD-2HA or 2HA-GRD-C10 constructs at MOI 5000. After 4 days, viable cells were measured. Statistical significance was evaluated by two-tailed t-test. NS: not significant, p>0.05.

FIG. 6A is a schematic drawing showing the C-terminal hypervariable regions (HVRs) of human HRas (SEQ ID NO: 21), NRas (SEQ ID NO: 22), KRas4A (SEQ ID NO: 23), and KRas4B (SEQ ID NO: 24) in fusion with the NF1-GRD. The C-terminal shaded four amino acids are the CAAX motifs, with prenylation sites (cysteine) marked by a black arrow head. The purported palmitoylation sites (cysteine) are boxed and the polybasic residues are underlined in solid lines. The basic/hydrophobic sequences are underlined in dashed lines. These elements all contribute to the targeting of proteins to the cellular membrane.

FIG. 6B is an immunoblot showing protein expression in the ipn02.3-2A, ipNF9511bc, and ST8814 cell lines used during experiments described herein. FIG. 7A is a bar plot showing the inhibition of cell growth in the NF-1 plexiform ipNF9511bc cell line by GRD constructs fused with C-terminal Ras HVR sequences and transduced by AAV-DJ at MOI 5000, 500, or 100.

FIG. 7B is a bar plot showing the inhibition of cell growth in human ST8814 MPNST cell line by GRD constructs fused with C-terminal Ras HVR sequences and transduced by AAV-DJ at MOI 500. The data show that the KRas4B C-terminal sequence is more potent than HRas-C10 in fusion with GRD in inhibiting NF-1 cell lines.

FIG. 8 is a bar plot showing the inhibition of cell growth in the NF-1 plexiform ipNF9511bc cell line by GRD constructs fused with C-terminal Ras HVR sequences and transduced by AAV-DJ at MOI 5000, 500, or 100.

FIG. 9 is a bar plot showing the inhibition of cell growth in human ST8814 MPNST cell line by GRD constructs fused with C-terminal Ras HVR sequences and transduced by AAV-DJ at MOI 5000, 500, or 100.

FIG. 10 is a series of fluorescence microscope images showing the diffused cellular distribution of GRD and membrane targeting of the GRD-Kras4B-C24 fusion protein in ipNF9511bc cells.

FIG. 11 is an image of immunoblots showing the transduction of GRD-2HA and 2HA-GRD-KRas4B-C24 in ipNF9511bc cells by AAV-DJ. ipNF9511bc cells were transduced at MOI 5000 by AAV-DJ carrying EGFP (control), GRD-2HA, or 2HA-GRD-KRas4B-C24. The anti-HA western blot showed the expression of GRD-2HA and 2HA-GRD-KRas4B-C24, and the anti-pERK1/2 blot demonstrated the complete inhibition of ERK1/2 (p42/44) phosphorylation, a major downstream target of Ras pathway, by the 2HA-GRD-KRas4B-C24 construct, more effectively than by GRD-2HA. The anti-ERK1/2 blot showed the total levels of ERK1/2 in the lysates, whereas the anti-beta-actin blot was used as a control.

FIG. 12A is an alignment showing the homology shared by the CAAX-containing C-terminal HVR of HRas orthologs across different species (SEQ ID NOs: 25-34).

FIG. 12B is an alignment showing the homology shared by the CAAX-containing C-terminal HVR of NRas orthologs across different species (SEQ ID NOs: 35-44).

FIG. 12C is an alignment showing the homology shared by the CAAX-containing C-terminal HVR of KRas4A orthologs across different species (SEQ ID NOs: 45-54).

FIG. 12D is an alignment showing the homology shared by the CAAX-containing C-terminal HVR of KRas4B orthologs across different species (SEQ ID NOs: 55-64).

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to treatment of Ras-associated diseases and particularly, but not exclusively, to compositions, methods, systems, and kits for modulating (e.g., decreasing) Ras activity using a neurofibromin 1 GTPase-activating protein-related domain gene therapy construct.

Loss of functional neurofibromin 1 (NF1) protein in humans (e.g., as a result of particular genetic alterations) causes neurofibromatosis type 1, which includes the highly aggressive malignant peripheral nerve sheath tumors (MPNSTs). A major function of NF1 is suppressing Ras activity through the intrinsic GTPase-activating protein-related domain (GRD) of NF1. During the development of embodiments of the technology, experiments were conducted to modulate Ras activity by the exogenous expression of various GRD constructs. Furthermore, experiments were conducted to test delivery of a GRD construct using a panel of adeno-associated virus (AAV) vectors in MPNST and human Schwann cells (HSC). The data collected in these experiments demonstrated that several AAV serotypes achieved favorable transduction efficacies in those cells and a membrane-targeting GRD fused with Ras CAAX motif-containing C-terminal HVR sequences, such as HRas-C10 (GRD-C10) and -C24, NRas-C24, KRas4A C24, or KRas4B -C21 and -C24 dramatically inhibited the Ras pathway (exemplified by GRD-C10 and GRD-KRas4B-C24) and MPNST/NF-1 cells in a NF1-specific manner. Embodiments of the technology find use in providing a gene replacement therapy for treating NF1-related tumors.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

Ranges provided herein are understood to be shorthand for all values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “sequencing-free” method does not comprise a sequencing step, etc.

As used herein, an “increase” or a “decrease” refers to a detectable (e.g., measured) positive or negative change in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control.

Other terms indicating quantitative changes or differences, such as “more” or “less,” are used herein in the same fashion as described above.

As used herein, the term “Rasopathy” refers to a group of genetic syndromes caused by germline mutations in genes that encode components or regulators of the Ras/mitogen-activated protein kinase (MAPK) pathway. These syndromes include, e.g., neurofibromatosis type 1, Noonan syndrome, Noonan syndrome with multiple lentigines, capillary malformation-arteriovenous malformation syndrome, Costello syndrome, cardio-facio-cutaneous syndrome, and Legius syndrome. The Ras/MAPK pathway plays an essential role in regulating the cell cycle and cellular growth, differentiation, and senescence, all of which are critical to normal development. Because of the common underlying Ras/MAPK pathway dysregulation, the Rasopathies exhibit numerous overlapping phenotypic features. These overlapping phenotypes can in some cases exist or be caused by mechanisms that operate independently of MAPK itself.

As used herein, the term “sample” or “biological sample” refers to anything that may contain cells of interest (e.g., cancer or tumor cells thereof) for which a screening method or treatment is desired. The sample may be a biological sample, such as a biological fluid or a biological tissue. Such a sample may include diverse cells, proteins, and genetic material. Examples of biological tissues also include organs, tumors, lymph nodes, arteries, and individual cell(s). Examples of biological fluids include urine, blood, plasma, serum, saliva, semen, stool, sputum, cerebral spinal fluid, tears, mucus, amniotic fluid, or the like.

As used herein, the term “subject” or “patient” refers to a human or non-human mammal. Non-human mammals include, for example, livestock and pets, such as ovine, bovine, porcine, canine, feline, and murine mammals. In some embodiments, the subject is human.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a biological organism, e.g., a mammal, particularly in a human, and includes: (a) preventing the disease from occurring in a subject predisposed to the disease or at risk of acquiring the disease but has not yet been diagnosed as having it; (b) inhibiting the disease, e.g., arresting its development; and (c) relieving the disease, e.g., causing regression of the disease.

As used herein, the term “prevent” means to stop or hinder something from happening, especially by advance planning or action, or prophylactic treatment. Prevention implies anticipatory counteraction, such as by administering a therapeutic or a composition containing a therapeutic to a patient at risk of developing a disease that is preventable via the therapeutic.

As used herein, the phrase “prior to treatment” can refer to a time prior to the commencement of treatment and can also refer to a time prior to the current treatment. That is, “prior to treatment” can refer to a prior treatment.

As used herein, the terms “pharmaceutically acceptable” and “physiologically acceptable” mean a biologically acceptable formulation, gaseous, liquid or solid, or mixture thereof, suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a AAV vector or AAV virion as disclosed herein or transformed cell to a subject.

As used herein, the term “unit dosage form” refers to physically discrete units suited as unitary dosages for the subject to be treated; each unit containing a predetermined quantity optionally in association with a pharmaceutical carrier (excipient, diluent, vehicle or filling agent) which, when administered in one or more doses, produces a desired effect (e.g., prophylactic or therapeutic effect). In some embodiments, unit dosage forms may be within, for example, ampules and vials, including a liquid composition, or a composition in a freeze-dried or lyophilized state; a sterile liquid carrier, for example, can be added prior to administration or delivery in vivo. Individual unit dosage forms can be included in multi-dose kits or containers. AAV vectors or AAV virions, and pharmaceutical compositions thereof, can be packaged in single or multiple unit dosage form for ease of administration and uniformity of dosage.

As used herein, the term “therapeutically effective amount” will fall in a relatively broad range determinable through experimentation and/or clinical trials. For example, in some embodiments, a therapeutically effective dose is on the order of from approximately 10⁶ to approximately 10¹⁵ AAV virions, e.g., from approximately 10⁸ to 10¹² AAV virions. For example, in some embodiments, a therapeutically effective dose is on the order of from approximately 10⁶ to approximately 10¹⁵ infectious units, e.g., from approximately 10⁸ to approximately 10¹² infectious units. Other effective dosages can be readily established by one of ordinary skill in the art through routine trials establishing dose response curves.

As used herein, the term an “effective amount” or “sufficient amount” refers to an amount providing, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

As used herein, the term “effective amount” or “sufficient amount” refer to doses for treatment (e.g., to ameliorate or to provide a therapeutic benefit or improvement) typically effective to provide a response to one, multiple, or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

As used herein, the term “prophylaxis” and grammatical variations thereof mean a method in which contact, administration, or in vivo delivery to a subject is prior to disease. Administration or in vivo delivery to a subject can be performed prior to development of an adverse symptom, condition, complication, etc. caused by or associated with the disease. For example, a screen (e.g., genetic) can be used to identify such subjects as candidates for the described methods and uses, but the subject may not manifest the disease. Such subjects therefore include those screened positive for an insufficient amount or a deficiency in a functional gene product (e.g., a protein (e.g., NF1)), or producing an aberrant (e.g., hyperactive), partially functional, or non-functional gene product (e.g., a protein (e.g., Ras)), leading to disease (e.g., a neurofibroma, neurofibromatosis, and/or a MPNST); and subjects screening positive for an aberrant, or defective (mutant) gene product (protein) leading to disease, even though such subjects do not manifest symptoms of the disease.

As used herein, “multiplicity of infection” (MOI) generally refers to the number of virions that are added per cell during infection.

As used herein, “operably linked” means incorporated into a genetic construct so that expression control sequences effectively control expression of a coding sequence of interest. The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of gene to a transcriptional control element refers to the physical and functional relationship between the gene and promoter such that the transcription of the gene is initiated from the promoter by an RNA polymerase that specifically recognizes, binds to and transcribes the DNA.

As used herein, the term “vector” or “construct” refers to a polynucleotide capable of transporting into a cell another polynucleotide to which the vector sequence has been linked. For example, in some embodiments, a “vector” is a replicon, such as a plasmid, phage, virus, or cosmid into which another DNA segment may be inserted to bring about the replication of the inserted segment. “Plasmid” and “vector” are used interchangeably, as a plasmid is a commonly used form of vector. Vectors can be expression vectors. An “expression vector” is a vector that includes one or more expression control sequences, and an “expression control sequence” is a DNA sequence that controls and regulates the transcription and/or translation of another DNA sequence. The term “expression vector” includes any vector, (e.g., a plasmid, cosmid, or phage chromosome) containing a gene construct in a form suitable for expression by a cell (e.g., linked to a transcriptional control element).

As used herein, the term “AAV” is an abbreviation for “adeno-associated virus” and may be used to refer to the virus itself or derivatives thereof. The term covers all subtypes and both naturally occurring and recombinant forms, except where required otherwise. The abbreviation “rAAV” refers to recombinant adeno-associated virus, also referred to as a recombinant AAV vector (or “rAAV vector”). The term “AAV” or “adeno-associated virus” includes AAV type 1 (AAV1), AAV type 2 (AAV2), AAV type 3 (AAV3), AAV type 4 (AAV4), AAV type 5 (AAVS), AAV type 6 (AAV6), AAV type 7 (AAV7), AAV type 8 (AAV8), AAV type 9 (AAV9), avian AAV, bovine AAV, canine AAV, equine AAV, primate AAV, non-primate AAV, and ovine AAV. “Primate AAV” refers to AAV capable of infecting primates, “non-primate AAV” refers to AAV capable of infecting non-primate mammals, “bovine AAV” refers to AAV capable of infecting bovine mammals, etc.

As used herein, the term “AAV vector” refers to an AAV vector nucleic acid sequence encoding for a variant capsid polypeptide (e.g., the AAV vector comprises a nucleic acid sequence encoding for a variant capsid polypeptide), wherein the variant capsid polypeptide is more cationic than a substantially identical a non-variant parent capsid polypeptide and/or another variant capsid polypeptide, and wherein said vector comprising said variant capsid polypeptide is capable of comprising a longer nucleic acid insert as compared to a vector comprising a non-variant parent capsid polypeptide and/or another variant capsid polypeptide. The AAV vectors can further comprise a heterologous nucleic acid sequence not of AAV origin (i.e., a nucleic heterologous to AAV), as part of the longer nucleic acid insert. This heterologous nucleic acid sequence typically comprises a sequence of interest for the genetic transformation of a cell. In general, the heterologous nucleic acid sequence is flanked by at least one, and generally by two AAV inverted terminal repeat sequences (ITRs).

As used herein, the term “AAV virion” or “AAV virus” or “AAV viral particle” or “AAV vector particle” refers to a viral particle comprising at least one AAV capsid polypeptide (including both variant capsid polypeptides and non-variant parent capsid polypeptides) and an encapsidated polynucleotide AAV vector. If the particle comprises a heterologous nucleic acid (e.g., a polynucleotide other than a wild-type AAV genome, such as a transgene to be delivered to a mammalian cell), it can be referred to as an “AAV vector particle” or simply an “AAV vector”. Thus, production of AAV virion or AAV particle necessarily includes production of AAV vector as such a vector is contained within an AAV virion or AAV particle.

As used herein, the term “packaging” refers to a series of intracellular events resulting in the assembly and encapsidation of an AAV virion or AAV particle.

As used herein, the terms “rep” and “cap” genes refer to polynucleotide sequences encoding replication and encapsidation proteins of adeno-associated virus. AAV rep (replication) and capsid (capsid) are referred to herein as AAV “packaging genes.”

As used herein, the term “helper virus” for AAV refers to a virus allowing AAV (e.g., wild-type AAV) to be replicated and packaged by a mammalian cell. A variety of such helper viruses for AAV are known in the art, including adenoviruses, herpesviruses, and poxviruses such as vaccinia. The adenoviruses comprise a number of different subgroups, although Adenovirus type 5 of subgroup C is most commonly used as a helper virus. Numerous adenoviruses of human, non-human mammalian, and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV), which are also available from depositories such as ATCC.

As used herein, the term “helper virus function(s)” refers to function(s) encoded in a helper virus genome allowing AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The terms “transformation” and “transfection” refer to the introduction of a polynucleotide, e.g., an expression vector, into a recipient cell including introduction of a polynucleotide to the chromosomal DNA of the cell.

As used herein, the terms “polypeptide,” “peptide,” and “protein” are used interchangeably herein to refer to polymers of amino acids of any length. The “polypeptides”, “proteins”, and “peptides” encoded by the “polynucleotide sequences” include full-length native sequences, as with naturally occurring proteins, as well as functional subsequences, modified forms or sequence variants so long as the subsequence, modified form, or variant retains some degree of functionality of the native full-length protein. In methods and uses of polypeptides and polynucleotides encoding polypeptides as described herein, such polypeptides, proteins, and peptides encoded by the polynucleotide sequences can be, but are not required to be, identical to a defective endogenous protein, or whose expression is insufficient, or deficient in a treated mammal. The terms also encompass a modified amino acid polymer, e.g., disulfide bond formation, glycosylation, lipidation, prenylation, palmitoylation, phosphorylation, or conjugation with a labeling component. Polypeptides such as NF1 polypeptides and GRD polypeptides, when discussed in the context of delivering a gene product to a mammalian subject, and compositions therefor, refer to the respective intact polypeptide, or any fragment or genetically engineered derivative thereof, retaining the desired biochemical function of the intact protein.

As used herein, the term “residue” refers to an amino acid that is incorporated into a protein. The amino acid may be a naturally occurring amino acid and, unless otherwise limited, may encompass known analogs of natural amino acids that can function in a similar manner as naturally occurring amino, acids. Conventional one and three-letter amino acid codes are used herein as follows—Alanine: Ala, A; Arginine: Arg, R; Asparagine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine: Val, V. As used herein, the codes Xaa and X refer to any amino acid.

As used herein, the term “CAAX” refers to an amino acid sequence that comprises a cysteine (C) followed by any two aliphatic amino acids (A), followed by any amino acid. As used herein, the term “aliphatic amino acid” refers to an amino acid comprising an aliphatic hydrocarbon side chain (e.g., an amino acid that is nonpolar and/or hydrophobic), e.g., D- or L-isomers of leucine, valine, norvaline, norleucine, and isoleucine. In some embodiments, glycine, alanine, proline, and methionine are considered to be aliphatic amino acids.

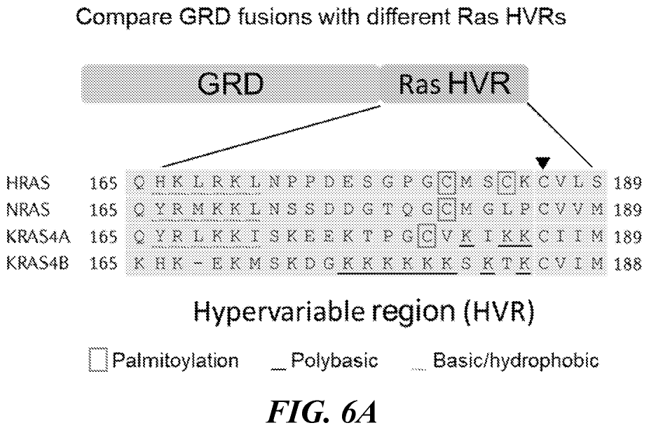

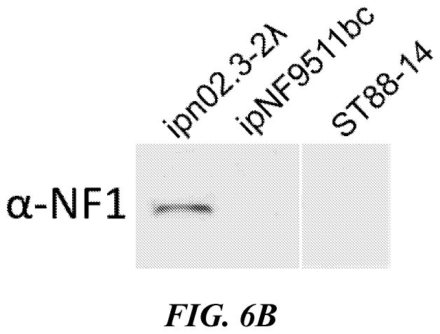

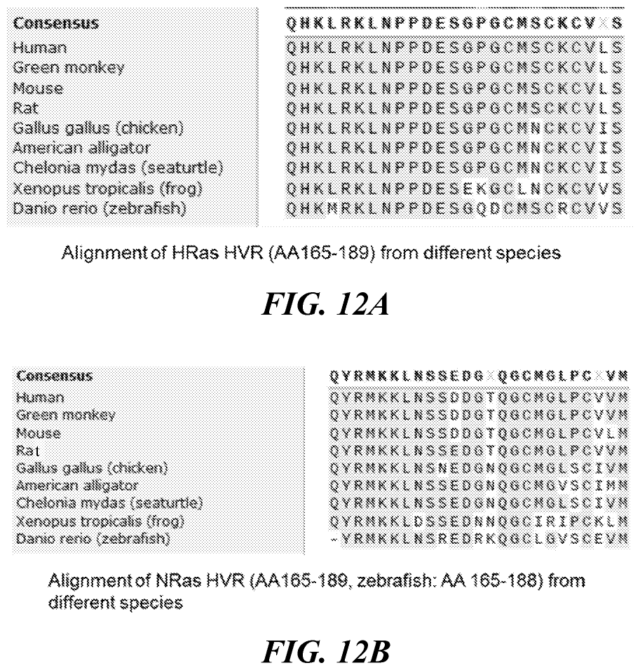

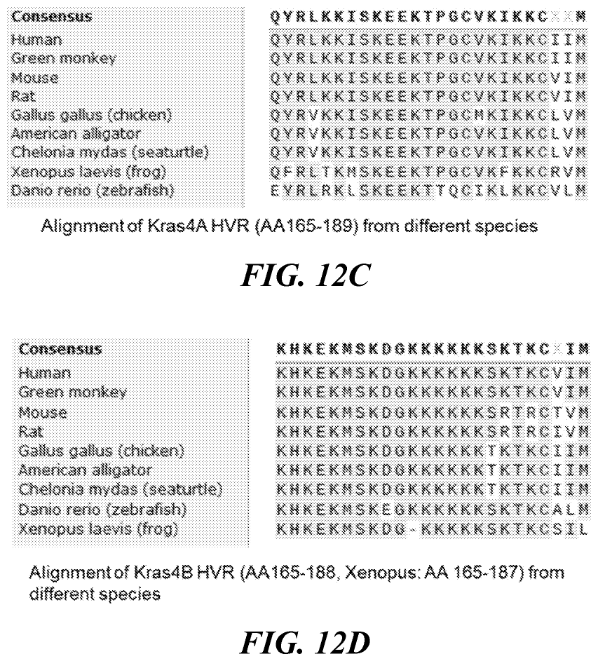

As used herein, the term “HVR” (hypervariable region) refers to an amino acid sequence that is located at the C-terminal membrane targeting region of a Ras protein. The HVR is outside of the G domain of a Ras protein and is commonly referred to as the “hypervariable region” [43]. During the development of embodiments of the technology provided herein, experiments were conducted using polypeptides comprising a human Ras HVR sequence (see, e.g., FIG. 6A). However, the technology is not limited to use of a human Ras HVR and the technology comprises use of an HVR amino acid sequence from any species, e.g., a Ras HVR from mouse, rat, xenopus, and/or Danio rerio, all of which share significant homology and convey the same membrane-targeting activity as human Ras HVR (see, e.g., FIGS. 12A-12D and [44] (incorporated herein by reference)), and/or a polypeptide having the membrane-targeting activity of an HVR. Accordingly, the term “HVR” refers to an HVR from any species and/or any polypeptide sequence having activity of an HVR, whether obtained from a Ras homolog and/or comprising one or more substitutions relative to a Ras homolog. In some embodiments, an HVR comprises amino acids 165-189 of the amino acid sequence of HRas, NRas, or KRas4A. In some embodiments, an HVR comprises amino acids 165-188 of KRas4B. In some embodiments, an HVR comprises the CAAX motif at the C-terminus, palmitoylation-targeted cysteine residues and/or polybasic residues, and/or as hydrophobic residues for membrane-targeting. Further, as used herein, a “CAAX motif-containing partial or complete Ras HVR sequence” is an amino acid sequence from a Ras HVR that comprises a CAAX motif (e.g., a partial or complete Ras HVR sequence (e.g., a sequence comprising a CAAX motif)).

As used herein, the term “tag” refers to an amino acid sequence that is, in some embodiments, fused to or included in the amino acid sequence of a polypeptide, e.g: a) improving expression of the polypeptide; b) facilitating purification of the polypeptide; facilitating immobilization of the polypeptide; and/or d) facilitating detection of the polypeptide. Examples for tags are His tags (e.g., 5× His-tags, 6× His-tags, 7× His-tags, 8× His-tags, 9× His-tags, 10× His-tags, 11× His-tags, 12× His-tags, 16× His-tags, 20× His-tags), Strep-tags, Avi-tags, Myc-tags, GST-tags, JS-tags, cystein-tags, FLAG-tags, HA-tags, thioredoxin, or maltose binding proteins (MBP), CAT, GFP, YFP, etc. The person skilled in the art knows a vast number of tags suitable for different technical applications. The tag may, for example, make a tagged polypeptide suitable for, e.g., antibody binding in different ELISA assay formats or other technical applications. The technology described herein is not limited to polypeptides comprising a tag. For example, therapeutic embodiments may not comprise a tag and embodiments used for experimentation and research may comprise a tag.

As used herein, the term “variant” refers to an amino acid sequence or nucleic acid sequence having conservative substitutions, non-conservative substitutions (that is, a degenerate variant), substitutions within the wobble position of a codon encoding an amino acid, amino acids added to the C-terminus of a peptide, or a peptide having 60%, 70%, 80%, 90%, 95%, 96%, 97%, 98%, or 99% sequence identity to an amino acid sequence.

As used herein, the term “conservative variant” refers to a particular nucleic acid sequence that encodes identical or essentially identical amino acid sequences.

Conservative substitution tables providing functionally similar amino acids are well known in the art. The following sets forth exemplary groups which contain natural amino acids that are “conservative substitutions” for one another: Alanine (A), Serine (5), and Threonine (T); Aspartic acid (D) and Glutamic acid (E); Asparagine (N) and Glutamine (Q); Arginine (R) and Lysine (K); Isoleucine (I), Leucine (L), Methionine (M), and Valine (V); and Phenylalanine (F), Tyrosine (Y), and Tryptophan (w).

As used herein, the term “percent (%) sequence identity” or “homology” refers to the percentage of nucleotides or amino acids in a candidate sequence that are identical with the nucleotides or amino acids in a reference nucleic acid or amino acid sequence after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2, or Megalign (DNASTAR) software. Appropriate parameters for measuring alignment, including any algorithms needed to achieve maximal alignment over the full-length of the sequences being compared, can be determined by known methods.

As used herein, the terms “polynucleotide” and “nucleic acid” are used interchangeably to refer to all forms of nucleic acid, including deoxyribonucleic acid (DNA) and ribonucleic acid (RNA). Polynucleotides include genomic DNA, cDNA, and antisense DNA, and spliced or unspliced mRNA, rRNA, tRNA, lncRNA, RNA antagomirs, and inhibitory DNA or RNA (RNAi, e.g., small or short hairpin (sh)RNA, microRNA (miRNA), aptamers, small or short interfering (si)RNA, trans-splicing RNA, or antisense RNA). Polynucleotides also include non-coding RNA, which include for example, but are not limited to, RNAi, miRNAs, lncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double-stranded and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction. As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example, a 24-residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

It is well known that DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. It is also known that all of these 5 types of nucleotides specifically bind to one another in combinations called complementary base pairing. That is, adenine (A) pairs with thymine (T) (in the case of RNA, however, adenine (A) pairs with uracil (U)), and cytosine (C) pairs with guanine (G), so that each of these base pairs forms a double strand. Conventional codes are used herein as follows—R (G or A), Y (T/U or C), M (A or C), K (G or T/U), S (G or C), W (A or T/U), B (G or C or T/U), D (A or G or T/U), H (A or C or T/U), V (A or G or C), or N (A or G or C or T/U), gap (-).

As used herein, the term “recombinant”, as applied to a polynucleotide, means the polynucleotide is the product of various combinations of cloning, restriction, or ligation steps, and other procedures resulting in a construct distinct and/or different from a polynucleotide found in nature. A recombinant virus is a viral particle comprising a recombinant polynucleotide. The terms respectively include replicates of the original polynucleotide construct and progeny of the original virus construct.

As used herein, the term “gene” refers to a polynucleotide that encodes a protein or functional RNA molecule. For instance, the term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “Ras pathway” or “Ras/Raf/MEK/ERK pathway” refers to the intracellular kinase cascade comprising RAS, RAF, mitogen-activated protein kinase kinase (MEK), and extracellular signal regulated kinase (ERK). The Ras/Raf/MEK/ERK signaling pathway is known in the art and described in McCormick (1993) Nature 363 (6424): 15-6, incorporated herein by reference. As used herein, an “activated Ras/Raf/MEK/ERK pathway” refers to a detectable increase in levels of phosphorylated MAPK, and/or increased expression and/or activation of its primary targets (Favres (2006) Bulletin du Cancer 93(4): 25-30; Kolch (2000) Biochem J 351(2): 289-305; Peysonnaux and Eychece (2001) Biol Cell 93 :53-62; Satyamoorthy et al. (2003) Cancer Res 63: 756-759; Houben et al. (2004) J Carcinog 3: 6, each of which is incorporated herein by reference). An “activated Ras/Raf/MEK/ERK pathway is also defined as an increase in kinase activity of members of the pathway. Methods for measuring kinase activity of Ras/Raf/MEK/ERK pathway members are taught, for example, in Davies et al. (2002), Nature 417: 949, incorporated herein by reference.

The “mitogen-activated protein kinase”/ “extracellular signal-regulated kinase” (MAPK/ERK, or just MAPK) pathways are signal transduction pathways that couple intracellular responses to the binding of growth factors (such as EGF) to cell surface receptors (such as EGFR). The MAPK pathways are one of the major downstream pathways controlling cellular processes associated with fibrosis, including cell growth, proliferation, differentiation, migration, protection from apoptosis, and transformation. There are several distinct MAPK pathways, important in the regulation of cell proliferation, differentiation, development, inflammation, survival, and migration.

In transformed cells, the Ras-Raf-MEK-ERK pathway has been implicated in cell proliferation and survival. The Ras-Raf-MEK-ERK pathway is activated by a range of growth factor receptors (including EGFR, platelet-derived growth factor receptor, type-1 insulin-like growth factor receptor, and fibroblast growth factor receptor). The pathway can also be activated by cytokines, steroid hormones, and several agonists that act via G-protein-coupled receptors.

Growth-factor stimulation of the MAPK pathways, such as by EGF or TGF-α, leads to sequential activation of Ras and Raf, which in turn activate MAPK kinases 1 and 2 (MEK1 and MEK2, or together, MEK1/2). MEK1/2 is a dual-specificity kinase that phosphorylates mitogen-activated protein kinases (MAPKs) and extracellular signal-related kinases (ERKs). MEK1/2 is essential to the propagation of growth factor signaling and is known to amplify signals to extracellular signal-regulated kinases 1 and 2 (ERK1/2, also known as MAPK1/2). MAPKs are part of a major signal transduction route that, upon activation, can phosphorylate a variety of intracellular targets including transcription factors, transcriptional adaptor proteins, membrane and cytoplasmic substrates, and other protein kinases. MAPKs transfer and amplify messages from the cell surface to the nucleus, producing a range of cellular effects, including cell proliferation.

As used herein, the terms “modulate” or “modulates” with respect to Ras activity includes any measurable alteration, either a decrease (e.g., inhibition) or increase (e.g., enhancement), of Ras activity.

As used herein, the terms “inhibit” or “inhibits” with respect to Ras activity includes any measurable decrease of Ras activity.

As used herein, the terms “detect”, “detecting” or “detection” may describe either the general act of discovering or discerning or the specific observation of a detectably labeled composition.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

### Description

Provided herein is technology relating to treatment of Ras-associated diseases and particularly, but not exclusively, to compositions, methods, systems, and kits for decreasing Ras activity using a neurofibromin 1 GTPase-activating protein-related domain (NF1-GRD) gene therapy construct. Although the disclosure herein refers to certain illustrated embodiments, it is to be understood that these embodiments are presented by way of example and not by way of limitation.

### Neurofibromatosis 1 and Malignant Peripheral Nerve Sheath Tumors

Based on NIH clinical consensus criteria, the main clinical features of neurofibromatosis 1 include multiple café-au-lait (CAL) macules, axillary freckling, Lisch nodules, optic pathway gliomas, peripheral nerve-sheath tumors, various forms of cognitive dysfunction, and a higher risk of cancers such as pheochromocytoma and breast cancer in young women [2, 5]. Approximately 50% of individuals with neurofibromatosis 1 develop plexiform neurofibromas (pNFs), which are benign pre-cancerous tumors arising in the peripheral nerve and involve multiple nerve fascicles typically growing along a nerve and its branches and frequently colliding into large masses in plexi such as the brachial or lumbosacral plexi [2, 6]. Other tumors commonly associated with neurofibromatosis type 1 include malignant peripheral nerve sheath tumor (MPNST), central nervous system tumors (e.g., optic pathway glioma, astrocytoma, and brain stem glioma), soft tissue sarcoma, pheochromocytoma, and rhabdomyosarcoma. Gastrointestinal stromal tumors are also common in patients with neurofibromatosis type 1, usually occurring in multiple locations in the small intestines.

Approximately 8 to 13% of neurofibromatosis 1 patients develop an MPNST that evolves from a pre-existing pNF (˜80%), while a small portion arises de novo (˜20%) [2]; in patients with microdeletion the MPNST risk increases to 30-40%. MPNST is a highly aggressive spindle cell sarcoma that is the most common malignancy and the leading cause of death in individuals with neurofibromatosis 1, particularly for those younger than 40 years of age [7, 8]. Biallelic mutations that inactivate the NF1 gene in Schwann cells initiate the carcinogenesis of MPNSTs. The complete loss of function or substantial reduction of functional NF1, in combination with other accumulated genetic alterations (e.g., deletion of cell cycle regulator CDKN2A/B and loss-of-function mutations in the tumor suppressor TP53) and/or epigenetic alternations (e.g., loss-of-function of the histone methyltransferase polycomb repressive complex 2 (PRC2)), affect the regulation of multiple cellular processes, such as growth factor signaling, metabolism, and apoptosis over time [2, 9].

There is currently no consensus on the treatment standard for MPNSTs and the role of traditional cytotoxic chemotherapies, radiation, and radical surgical approaches (e.g., amputation) are not completely defined [2]. To date, surgery is the only treatment modality indicated by the data to provide a survival benefit for MPNSTs. However, even when maximal surgery with wide surgical margins is feasible, these tumors are almost never curable [8, 10]. A number of neurofibromatosis and sarcoma centers in the US utilize pre-operative and/or post-operative chemotherapy (e.g., comprising administration of ifosfamide or doxorubicin) in their treatment protocols with disease stabilization for the majority of patients and partial responses in a small fraction of patients [2, 11]. Even with the most aggressive standard interventions, there is low chance of long-term survival and approximately 60-80% of patients develop recurrences within 5 years after initial diagnosis, thus reflecting the urgent need for new and more effective therapeutics for this cancer.

Advancements have been made in understanding the signaling mechanisms and molecular networks of MPNSTs that have identified novel oncogenic targets and inspired a number of new therapeutic approaches. Yet, effective drugs that directly inhibit Ras are lacking. Therefore, translational research has focused on critical downstream effectors of Ras, e.g., MEK1/2 inhibitors. Preclinical data with a pharmacological MEK inhibitor PD0325901 reduced tumor growth and prolonged survival in a NF1 mouse model (NF1(fl/fl); Dhh-Cre) and NF-1 patient-derived MPNST cell xenografts [12]. However, similar to many other investigational treatments, no cures have been achieved [2]. Furthermore, modulating targets downstream of Ras may suffer the risk of resistance development by activating alternative pathways and ultimately limiting the therapeutic potential of targeting non-Ras targets and/or activities.

Chemopreventative strategies to reduce or completely prevent the risk of MPNST development have also been investigated [2]. However, in contrast to other tumor predisposition syndromes, neurofibromatosis 1 is a highly variable disease and considerable differences in clinical symptoms are noted even within the same family with the same mutation resulting in an often unexpected and unpredictable MPNST development that is thus not suitable for chemoprevention, apart from the fact that no such therapy exists to date.

Interestingly, gene therapy remains a largely unexplored technology for the treatment of NF1-related MPNSTs despite a known uniform monogenic alteration as underlying cause of tumor formation. In particular, the delivery vectors based on adeno-associated viruses (AAVs) have emerged as safe and effective, achieving clinically meaningful long-term gene expression, and have led to regulatory approval for some conditions. The recombinant AAV used for gene-therapy is a non-pathogenic, non-replicating parvovirus because rep and cap genes are cloned in a trans-plasmid without inverted terminal repeats (ITR). Unlike lentivirus, it has a reduced cancerogenicity because it rarely integrates into the genome of the host cell. Recombination AAV is able of infecting both dividing and quiescent cells with a low host immune recognition. To date more than 100 natural occurring human and nonhuman primate AAV serotypes have been identified [13]; however, because natural occurring viruses are not optimized for the delivery needs of genetic information, various approaches have been developed to overcome certain shortcomings in viral vector properties that have led to the creation of a vast number of engineered AAVs depending on the disease properties. With the progress made in recombinant techniques, a variety of approaches can be used to adapt the viral characteristics for the intended need.

Advancements in our understanding of AAV capsid structure have facilitated the rational design of AAV capsids to restrict or re-direct viral tropism and transduction, and considerable progress in both AAV capsid library development and screening methodology has enabled directed evolution of AAV capsids, which will ultimately ensure that transgene expression is reproducible, robust, occurs over an extended period and helps avoid activating the innate and adaptive immune system [13-15].

Gene delivery vectors based on AAVs have a packaging capacity of up to approximately 4.7 kb at near wild-type titers and infectivity, beyond which size packaging efficiency markedly decreases, and genomes with 5′ truncations become encapsidated [14]. The NF1 cDNA measures 8.5 kb and is consequently too large for AAV vectors. Accordingly, in some embodiments, the technology provided herein comprises use of the NF1-GAP related domain (NF1-GRD) of NF1, which is sufficient to deactivate Ras activity by accelerating the hydrolysis of GTP to a GDP [16, 17] and which is encoded by a portion of the NF1 gene comprising approximately 1 kb. The nucleic acid encoding the NF1-GRD is thus of an appropriate size for efficient packaging into AAV. Thus, in some embodiments, the technology comprises use of AAV-based delivery of NF1-GRD to downregulate Ras activity and “correct” the tumorigenic potential of affected cells. In some embodiments, the technology provides a gene therapy treatment for neurofibromatosis 1 comprising treating with a recombinant AAV comprising NF1-GRD.

See also, Hirbe (2014) “Neurofibromatosis type 1: a multidisciplinary approach to care” Lancet Neurol 13: 834-43, incorporated herein by reference in its entirety, discussing symptoms, diagnosis, and treatments.

### Compositions

In some embodiments, the technology provides compositions. For example, some embodiments provide a nucleic acid comprising a GRD domain of NF1 (“NF1-GRD”). In some embodiments, the nucleic acid comprises sufficient coding sequence to produce (e.g., by translation) a polypeptide comprising GTPase activity (e.g., GTPase activity of the GRD domain of NF1). In some embodiments, the nucleic acid comprises sufficient coding sequence to produce (e.g., by translation) a polypeptide comprising GTPase activity that inhibits Ras. In some embodiments, the nucleic acid comprises fewer than approximately 5 kb of nucleotides (e.g., having a size of less than 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5 kb).

In some embodiments, the nucleic acid comprises nucleotide sequence encoding the GRD domain of NF1 as known in the art (see, e.g., Reference 39 and/or NCBI Reference Sequence: NP_000258.1, each of which is incorporated herein by reference). In some embodiments, the nucleic acid comprises a nucleotide sequence encoding the amino acids from approximately amino acid 1150 to approximately amino acid 1600 of the NF1 protein amino acid sequence as provided by SEQ ID NO: 2 and/or NCBI Accession NP_000258.1. In some embodiments, the nucleic acid comprises a nucleotide sequence encoding approximately amino acid 1150 (e.g., approximately amino acid 1150 to approximately amino acid 1200 (e.g., amino acid 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200)) to approximately amino acid 1550 (e.g., approximately amino acid 1500 to approximately amino acid 1550 (e.g., amino acid 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, or 1550)) of the NF1 protein provided by SEQ ID NO: 2 and/or NCBI Accession NP_000258.1.

In some embodiments, the nucleic acid comprises a nucleotide sequence provided by SEQ ID NO: 1 and/or NCBI Accession NM_000267.3. In some embodiments, the nucleic acid comprises a nucleotide sequence provided by nucleotides 158 to 8613 of SEQ ID NO: 1. In some embodiments, the nucleic acid comprises a nucleotide sequence provided by approximately nucleotide 3448 to approximately nucleotide 4650 of SEQ ID NO: 1. In some embodiments, the nucleic acid comprises a nucleotide sequence provided by the sequence beginning within the range of approximately nucleotide 3448 to 3600 (e.g., 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, or 3600) and ending within the range of approximately nucleotide 4498 to 4650 (e.g., 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554, 4555, 4556, 4557, 4558, 4559, 4560, 4561, 4562, 4563, 4564, 4565, 4566, 4567, 4568, 4569, 4570, 4571, 4572, 4573, 4574, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4601, 4602, 4603, 4604, 4605, 4606, 4607, 4608, 4609, 4610, 4611, 4612, 4613, 4614, 4615, 4616, 4617, 4618, 4619, 4620, 4621, 4622, 4623, 4624, 4625, 4626, 4627, 4628, 4629, 4630, 4631, 4632, 4633, 4634, 4635, 4636, 4637, 4638, 4639, 4640, 4641, 4642, 4643, 4644, 4645, 4646, 4647, 4648, 4649, or 4650) of SEQ ID NO: 1.

In some embodiments, a nucleic acid comprising a nucleotide sequence encoding a NF1-GRD domain further comprises a nucleotide sequence encoding an optional tag, e.g., a hemagglutinin tag, e.g.:

SEQ ID NO: 4
TAC CCA TAC GAT GTT CCA GAT TAC GCT;
or
SEQ ID NO: 5
TAT CCA TAT GAT GTT CCA GAT TAT GCT

In some embodiments, a nucleic acid encodes a NF1-GRD domain comprising a hemagglutinin tag at its N-terminus; in some embodiments, a nucleic acid encodes a NF1-GRD domain comprising a hemagglutinin tag at its C-terminus.

Accordingly, in some embodiments, the technology provides compositions comprising a NF1-GRD polypeptide. In some embodiments, the technology provides compositions comprising a NF1-GRD polypeptide further comprising a tag, e.g., a hemagglutinin polypeptide tag at the N-terminus or C-terminus. In some embodiments, the amino acid sequence of the optional hemagglutinin polypeptide tag is provided by:

SEQ ID NO: 6
YPYDVPDYA

Hemagglutinin tags are known in the art, e.g., as discussed in Field et al. (1988) “Purification of a RAS-responsive adenylyl cyclase complex from Saccharomyces cerevisiae by use of an epitope addition method” Mol. Cell. Biol. 8: 2159-65, incorporated herein by reference.

In some embodiments, a nucleic acid comprising a nucleotide sequence encoding a NF1-GRD domain further comprises a nucleotide sequence encoding a plurality of hemagglutinin tags in a tandem array, e.g., having amino acid sequences of YPYDVPDYA (HA) (SEQ ID NO: 6); YPYDVPDYA YPYDVPDYA (2× HA) (SEQ ID NO: 7); YPYDVPDYA YPYDVPDYA YPYDVPDYA (3× HA) (SEQ IDNO: 8); YPYDVPDYA YPYDVPDYA YPYDVPDYA YPYDVPDYA (4× HA) (SEQ ID NO: 9); etc. In some embodiments, N× (e.g., 2×, 3×, 4×, etc.) hemagglutinin tags are provided at the C-terminus of a NF1-GRD polypeptide and/or at the N-terminus of a NF1-GRD polypeptide. Embodiments provide nucleic acids comprising nucleotide sequences encoding N× tandem arrays of hemagglutinin tags (e.g., a nucleic acid comprising the appropriate number of repeats of SEQ ID NO: 4 and/or SEQ ID NO: 5).

Accordingly, in some embodiments, the technology provides compositions comprising a NF1-GRD polypeptide comprising a plurality (e.g., 2×, 3×, 4×, etc.) of hemagglutinin polypeptide tags (e.g., as provided by SEQ ID Nos: 6, 7, 8, and/or 9) in tandem at the N-terminus or C-terminus of the NF1-GRD amino acid sequence.

In some embodiments, a nucleic acid comprising a nucleotide sequence encoding a NF1-GRD domain further comprises a nucleotide sequence encoding a CAAX motif-containing partial or complete Ras HVR amino acid sequence, e.g., an amino acid sequence provided by:

SEQ ID NO: 3
GCMSCKCVLS

Accordingly, in some embodiments the technology provides a polypeptide comprising a NF1-GRD domain and an amino acid sequence comprising a CAAX motif-containing partial or complete Ras HVR sequence, e.g., a sequence from a Ras HVR (e.g., a partial or complete Ras HVR sequence (e.g., a sequence comprising a CAAX motif)) (e.g., as provided by SEQ ID NO: 3 and detailed in FIG. 6A). The technology is not limited in the amino acid sequence of the CAAX motif-containing partial or complete Ras HVR and encompasses any amino acid sequence that provides a CAAX motif-containing partial or complete Ras HVR and any nucleotide sequence encoding a CAAX motif-containing partial or complete Ras HVR amino acid sequence. For example, the technology encompasses any amino acid sequence comprising a cysteine followed by any aliphatic amino acid, followed by another aliphatic amino acid, followed by any amino acid. The technology encompasses any nucleotide sequence that encodes an amino acid sequence comprising a cysteine followed by any aliphatic amino acid, followed by another aliphatic amino acid, followed by any amino acid. The technology is not limited in the amino acid sequence of palmitoylation residues and/or polybasic residues and encompasses any amino acid sequence that provides a palmitoylation sites and/or polybasic residues, and any nucleotide sequence encoding palmitoylation-targeted cysteine and polybasic amino acid sequence. The technology provides in various embodiments combinations of NF1-GRD, optional tags (e.g., HA tags), and a CAAX motif-containing partial or complete Ras HVR sequence, e.g., a sequence from a Ras HVR (e.g., a partial or complete Ras HVR sequence (e.g., a sequence comprising a CAAX motif)) with palmitoylation sites and/or polybasic sequence combined as described herein. For example, the technology comprises polypeptides, and nucleic acids encoding polypeptides, comprising a NF1-GRD domain; polypeptides, and nucleic acids encoding polypeptides, comprising a NF1-GRD domain and an optional tag (e.g., HA tag (e.g., HA tag, 2× HA tag, 3× HA tag, etc.)) for detection purposes; polypeptides, and nucleic acids encoding polypeptides, comprising a NF1-GRD domain and a CAAX motif-containing partial or complete Ras HVR sequence, e.g., a sequence from a Ras HVR (e.g., a partial or complete Ras HVR sequence (e.g., a sequence comprising a CAAX motif)) providing palmitoylation-targeted cysteines and/or polybasic amino acids; and polypeptides, and nucleic acids encoding polypeptides, comprising a NF1-GRD domain, an optional tag (e.g., HA tag (e.g., HA tag, 2× HA tag, 3× HA tag, etc.)), a CAAX motif-containing partial or complete Ras HVR. Embodiments provided that the optional tag (e.g., HA tag (e.g., HA tag, 2× HA tag, 3× HA tag, etc.)) is at the N-terminus or C-terminus of the NF1-GRD amino acid sequence, but only at the N-terminus of the NF1-GRD amino acid sequence when NF1-GRD is fused with the CAAX-containing partial or complete Ras HVR sequences at the C-terminus. The CAAX motif-containing partial or complete Ras HVR sequence providing palmitoylation-targeted cysteines and/or polybasic amino acids is at the C-terminus of NF1-GRD amino acid sequence.

The technology comprises variants of the nucleic acids and polypeptides above, e.g., comprising one or more nucleotide or amino acid substitutions. Embodiments provide nucleic acids having at least 75% (e.g., at least 75, 76, 77, 78, 79, 80, 81, 82, 83, 84, 85, 86, 87, 88, 89, 90, 91, 92, 93, 94, 95, 96, 97, 98, or 99%) identity to the nucleic acids described herein. Embodiments provide polypeptides having at least 75% (e.g., at least 75, 76, 77, 78, 79, 80, 81, 82, 83, 84, 85, 86, 87, 88, 89, 90, 91, 92, 93, 94, 95, 96, 97, 98, or 99%) identity to the polypeptides described herein. Embodiments provide nucleic acids having at least 75% identity (e.g., at least 75, 76, 77, 78, 79, 80, 81, 82, 83, 84, 85, 86, 87, 88, 89, 90, 91, 92, 93, 94, 95, 96, 97, 98, or 99%) to the nucleic acids and/or polypeptides described herein provided that the variant nucleic acid or polypeptide functions as described herein.

Additionally, in some embodiments, the technology provides vectors comprising nucleic acids as described above. For example, in some embodiments, the technology provides an AAV vector comprising a nucleic acid as described above (e.g., an AAV vector comprising a nucleotide sequence encoding a polypeptide comprising a NF1-GRD domain; an AAV vector comprising a nucleotide sequence encoding a polypeptide comprising a NF1-GRD domain and an optional tag (e.g., HA tag (e.g., HA tag, 2× HA tag, 3× HA tag, etc.)); an AAV vector comprising a nucleotide sequence encoding a polypeptide comprising a NF1-GRD domain and a CAAX motif-containing partial or complete Ras HVR sequence, e.g., a sequence from a Ras HVR (e.g., a partial or complete Ras HVR sequence (e.g., a sequence comprising a CAAX motif)); and/or an AAV vector comprising a nucleotide sequence encoding a polypeptide comprising a NF1-GRD domain, a HA tag (e.g., HA tag, 2× HA tag, 3× HA tag, etc.), and a CAAX motif-containing partial or complete Ras HVR sequence, e.g., a sequence from a Ras HVR (e.g., a partial or complete Ras HVR sequence (e.g., a sequence comprising a CAAX motif))). In some embodiments, the AAV vector is AAV1 (e.g., pAAV²/₁), AAV2 (e.g., pAAV2-RC), AAV3B (e.g., pAAV-RC3B), AAV4 (e.g., pAAV-RC4), AAVS (e.g., pAAV2/5 JC), AAV6 (e.g., pAAV-RC6), AAV7 (e.g., pAAV2/7), AAV8 (e.g., pAAV2/8), AAV9 (e.g., pAAV9n), AAV10 (e.g., pAAV2/rh10), AAV11 (e.g., pAAV2/hull), AAV32/33 (e.g., pAAV2/rh32.33), or AAV-DJ (e.g., pAAVDJ). In some embodiments, the AAV vector is AAV2, AAV3B, AAV6, or AAV-DJ. See, e.g., References 21-25, each of which is incorporated herein by reference in its entirety.

Further embodiments provide a cell, a tissue, an organ, and/or an organism comprising a nucleic acid as described herein (e.g., an AAV vector as described above (e.g., a cell, a tissue, an organ, and/or an organism comprising an AAV vector comprising a nucleotide sequence encoding a polypeptide comprising a NF1-GRD domain; an AAV vector comprising a nucleotide sequence encoding a polypeptide comprising a NF1-GRD domain and an optional tag (e.g., HA tag (e.g., HA tag, 2× HA tag, 3× HA tag, etc.)); an AAV vector comprising a nucleotide sequence encoding a polypeptide comprising a NF1-GRD domain and a CAAX motif-containing partial or complete Ras HVR sequence, e.g., a sequence from a Ras HVR (e.g., a partial or complete Ras HVR sequence (e.g., a sequence comprising a CAAX motif)); and/or an AAV vector comprising a nucleotide sequence encoding a polypeptide comprising a NF1-GRD domain, an optional tag (e.g., HA tag (e.g., HA tag, 2× HA tag, 3× HA tag, etc.)), and a CAAX motif-containing partial or complete Ras HVR sequence, e.g., a sequence from a Ras HVR (e.g., a partial or complete Ras HVR sequence (e.g., a sequence comprising a CAAX motif))).

In some embodiments, the technology provides a cell comprising a nucleic acid (e.g., a nucleic acid comprising a nucleotide sequence encoding a polypeptide comprising a NF1-GRD domain; a nucleic acid comprising a nucleotide sequence encoding a polypeptide comprising a NF1-GRD domain and a HA tag (e.g., HA tag, 2× HA tag, 3× HA tag, etc.); a nucleic acid comprising a nucleotide sequence encoding a polypeptide comprising a NF1-GRD domain and a CAAX motif-containing partial or complete Ras HVR sequence, e.g., a sequence from a Ras HVR (e.g., a partial or complete Ras HVR sequence (e.g., a sequence comprising a CAAX motif)); and/or a nucleic acid comprising a nucleotide sequence encoding a polypeptide comprising a NF1-GRD domain, a HA tag (e.g., HA tag, 2× HA tag, 3× HA tag, etc.), and a CAAX motif-containing partial or complete Ras HVR sequence, e.g., a sequence from a Ras HVR (e.g., a partial or complete Ras HVR sequence (e.g., a sequence comprising a CAAX motif))) integrated into the chromosome (e.g., at the AAVS1 site of human chromosome 19). See, e.g., Kotin et al. (1990) “Site-specific integration by adeno-associated virus” Proceedings of the National Academy of Sciences of the United States of America. 87 (6): 2211-5; Surosky et al. (1997) “Adeno-associated virus Rep proteins target DNA sequences to a unique locus in the human genome” Journal of Virology. 71 (10): 7951-9, each of which is incorporated herein by reference.

In some embodiments, the technology provides a tissue, organ, and/or organism comprising a cell comprising a nucleic acid (e.g., a nucleic acid comprising a nucleotide sequence encoding a polypeptide comprising a NF1-GRD domain; a nucleic acid comprising a nucleotide sequence encoding a polypeptide comprising a NF1-GRD domain and a HA tag (e.g., HA tag, 2× HA tag, 3× HA tag, etc.); a nucleic acid comprising a nucleotide sequence encoding a polypeptide comprising a NF1-GRD domain and a CAAX motif-containing partial or complete Ras HVR sequence, e.g., a sequence from a Ras HVR (e.g., a partial or complete Ras HVR sequence (e.g., a sequence comprising a CAAX motif)); and/or a nucleic acid comprising a nucleotide sequence encoding a polypeptide comprising a NF1-GRD domain, a HA tag (e.g., HA tag, 2× HA tag, 3× HA tag, etc.), and a CAAX motif-containing partial or complete Ras HVR sequence, e.g., a sequence from a Ras HVR (e.g., a partial or complete Ras HVR sequence (e.g., a sequence comprising a CAAX motif))) integrated into the chromosome (e.g., at the AAVS1 site of human chromosome 19).

In some embodiments, the technology provides a cell expressing a polypeptide comprising a NF1-GRD domain. In some embodiments, the technology provides a cell expressing a polypeptide comprising a NF1-GRD domain joined to a CAAX motif-containing partial or complete Ras HVR sequence, e.g., a sequence from a Ras HVR (e.g., a partial or complete Ras HVR sequence (e.g., a sequence comprising a CAAX motif)). In some embodiments, the technology provides a tissue, organ, or organism comprising a cell expressing a polypeptide comprising a NF1-GRD domain and/or expressing a polypeptide comprising a NF1-GRD domain joined to a CAAX motif-containing partial or complete Ras HVR sequence, e.g., a sequence from a Ras HVR (e.g., a partial or complete Ras HVR sequence (e.g., a sequence comprising a CAAX motif)). In some embodiments, the cell is deficient in expressing NF1 (e.g., has a NF1 −/− or NF1 +/− genotype and/or has a decreased level of NF1 expression and/or a decreased level of NF1 activity (e.g., decreased GTPase activity)) and/or has aberrant (e.g., increased (e.g., tumor-forming)) Ras activity. In some embodiments, the cell is a MPNST cell. In some embodiments, the cell is a Schwann cell. In some embodiments, the cell has a mutation in the gene that encodes NF1 (e.g., a point mutation, inversion, deletion, insertion). In some embodiments, the cell has a mutation in a nucleotide sequence that controls expression of NF1. In some embodiments, the cell has a mutation in the gene that encodes Ras (e.g., a point mutation, inversion, deletion, insertion). In some embodiments, the cell has a mutation in a nucleotide sequence that controls expression of Ras. In some embodiments, a patient having a Rasopathy comprises the cell. In some embodiments, a patient having neurofibromatosis type 1 comprises the cell. In some embodiments, a patient having a MPNST comprises the cell.

The technology is not limited in the cells comprising the NF1-GRD nucleic acids or proteins or into which a NF1-GRD construct will be introduced. For example, cells encompassed by the technology provided herein include any cell into which foreign nucleic acids can be introduced and expressed as described herein. It is to be understood that the basic concepts of the present technology described herein are not limited by cell type. Cells according to the present disclosure include eukaryotic cells, mammalian cells, animal cells, human cells, and the like. Further, cells include any cells in which it would be beneficial or desirable to provide the NF1-GRD construct (e.g., nucleic acid and/or polypeptide). Such cells may include those that are deficient in expression of NF1 or having aberrant Ras activity (e.g., leading to a disease or detrimental condition). Such diseases or detrimental conditions are readily known to those of skill in the art, e.g., cancers (e.g., Rasopathies, neurofibroma, neurofibromatosis, malignant peripheral nerve sheath tumors). Providing the nucleic acid and polypeptide constructs described herein into such cells provide a therapeutic treatment in some embodiments.

### Pharmaceutical Compositions and Treatment

In some embodiments, a gene delivery vector (e.g., an AAV vector as described herein comprising a NF1-GRD construct), and packaged viral particles (e.g., AAV) comprising a viral vector, are provided in the form of a medicament or a pharmaceutical composition and are used in some embodiments in the manufacture of a medicament or a pharmaceutical composition. The pharmaceutical composition may comprise a pharmaceutically acceptable carrier. Preferably, the carrier is suitable for parenteral administration. In some embodiments, the carrier is suitable for intravenous, intraperitoneal, or intramuscular administration. Pharmaceutically acceptable carrier or excipients are described in, for example, Remington: The Science and Practice of Pharmacy, Alfonso R. Gennaro (Editor) Publishing Company (1997). Exemplary pharmaceutical composition embodiments comprise, e.g., sterile saline, dextrose solution, or buffered solution, or other pharmaceutically acceptable sterile fluids. Alternatively, a solid carrier may be used such as, for example, microcarrier beads.

Pharmaceutical compositions are typically sterile and stable under the conditions of manufacture and storage. Pharmaceutical compositions may be formulated as a solution, microemulsion, liposome, or other ordered structure suitable to delivery of the gene therapy vectors. The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. In many cases, embodiments comprise isotonic agents, for example, sugars, polyalcohols such as mannitol, sorbitol, or sodium chloride. Prolonged absorption of the injectable compositions can be brought about by including in the composition an agent that delays absorption, for example, monostearate salts and gelatin. The vectors of the present disclosure may be administered in a time or controlled release formulation, for example in a composition that includes a slow release polymer or other carriers that protect the compound against rapid release, including implants and microencapsulated delivery systems. Biodegradable, biocompatible polymers may, for example, be used, such as ethylene vinyl acetate, polyanhydrics, polyglycolic acid, collagen, polyorthoesters, polylactic acid, and polyglycolic copolymers (PLG).

In some embodiments, the gene therapy vectors, formulated with acceptable carriers, are administered parenterally, such as by intravenous, intraperitoneal, subcutaneous, intramuscular administration, limb perfusion, or combinations thereof. The administration can be systemic, such that the gene delivery vectors are delivered through the body of the subject. In some embodiments, the gene delivery vectors are administered directly into the targeted tissue, e.g., neural tissues. In some embodiments, the gene delivery vectors are administered locally, such as by a catheter. The route of administration can be determined by the person of skill in the art, taking into consideration, for example, the nature of target tissue, gene delivery vectors, intended therapeutic effect, and maximum load that can be administered and absorbed by the targeted tissue(s).

Generally, an effective amount, particularly a therapeutically effective amount, of the gene delivery vectors are administered to a subject in need thereof. An effective or therapeutically effective amount of vector may vary according to factors such as the disease state, age, sex, and weight of the individual, and the ability of the viral vector to elicit a desired response in the individual. Dosage regimens may be adjusted to provide the optimum therapeutic response.

In some embodiments, a range for therapeutically or prophylactically effective amounts of a nucleic acid, nucleic acid construct, AAV vector, or pharmaceutical composition may be from 1×10¹¹ and 1×10¹⁴ genome copy (gc)/kg or 1×10¹² and 1×10¹³ genome copy (gc)/kg. It is to be noted that dosage values may vary with the severity of the condition to be alleviated. The dosage may also vary based on the efficacy of the virion employed. For example, AAV8 is better at infecting liver as compared to AAV2 and AAV9 is better at infecting brain than AAV8, in these two cases one would need less AAV8 or AAV9 for the case of liver or brain respectively. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners.

In some embodiments, the effective dose range for small animals (mice), following intramuscular injection, may be between 1×10¹² and 1×10¹³ genome copy (gc)/kg, and for larger animals (cats or dogs) and for human subjects, between 1×10¹¹ and 1×10¹² gc/kg, or between 1×10¹¹ and 1×10¹⁴ genome copy (gc)/kg.

In various embodiments, the gene delivery vectors are administered as a bolus or by continuous infusion over time. In some embodiments, several divided doses are administered over time or the dose is proportionally reduced or increased as indicated by the exigencies of the therapeutic situation. In some embodiments, the gene delivery vectors can be administered daily, weekly, biweekly, or monthly. The duration of treatment can be for at least one week, one month, 2 months, 3 months, 6 months, or 8 months or more. In some embodiments, the duration of treatment can be for up to 1 year or more, 2 years or more, 3 years or more, or indefinitely.

### Methods

In some embodiments, a therapeutically effective amount of a gene delivery vector is administered to a subject to treat a condition or disease (e.g., a neurofibroma, neurofibromatosis type 1, and/or a MPNST). In some embodiments, the technology comprises administration for a period of time until an individual is generally healthy and free of disease and/or the methods ameliorate a disorder associated with a condition or disease (e.g., a neurofibromatosis type 1 and/or a MPNST).

In some embodiments, a gene therapy technology as described herein finds use in treating a subject having, suspected of having, predisposed to have, and/or in need of a treatment for, a cancer or benign tumor disease (e.g., a neurofibromatosis type 1 and/or a MPNST). For instance, in some embodiments, the technology reduces the incidence of disease and/or delays or ameliorates a disease. In some embodiments, the amelioration of disease provided by the gene therapy methods herein is a result of reducing symptoms in an affected subject or reducing the incidence of the disease or disorder in a population as compared to an untreated population. In some embodiments, the gene therapy has the effect of treating and/or preventing various benign tumor and/or cancer conditions and diseases as assessed by particular markers and disorders of said benign tumor and/or cancer conditions and diseases (e.g., genetic tests indicating NF1 mutations or Ras mutations; tests indicating aberrant NF1 and/or aberrant Ras activity). In a further aspect, therefore, the technology provides a gene therapy method or the use of a nucleic acid vector (e.g., AAV vector) as described above (e.g., comprising a nucleic acid encoding a NF1-GRD (e.g., comprising a nucleic acid encoding a NF1-GRD linked to a CAAX motif-containing partial or complete Ras HVR sequence, e.g., a sequence from a Ras HVR (e.g., a partial or complete Ras HVR sequence (e.g., a sequence comprising a CAAX motif)))) for use in the treatment or prevention in a subject of at least a disorder or marker of a cancer or benign tumor disease (e.g., a neurofibromatosis type 1 and/or a MPNST).

In some embodiments, the gene therapy described herein is used to extend the lifespan of a subject. Extended lifespan can be an increase in the average lifespan of an individual of that species who reaches adulthood and/or an extension of the maximum lifespan of that species. In some embodiments, extended lifespan can be a 5%, 10%, 15%, 20%, or more increase in maximum lifespan and/or a 5%, 10%, 15%, 20%, or more increase in average lifespan.

Foreign nucleic acids, alternatively referred to as heterologous nucleic acids (recombinant nucleic acids comprising a nucleotide sequence encoding a NF1-GRD or a NF1-GRD linked to a nucleotide sequence encoding a CAAX motif-containing partial or complete Ras HVR sequence, e.g., a sequence from a Ras HVR (e.g., a partial or complete Ras HVR sequence (e.g., a sequence comprising a CAAX motif)) as described herein) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources. Foreign nucleic acids may be delivered to a subject by administering to the subject, such as systemically administering to the subject, such as by intravenous administration or injection, intraperitoneal administration or injection, intramuscular administration or injection, intracranial administration or injection, intraocular administration or injection, subcutaneous administration or injection, a nucleic acid or vector including a nucleic acid as described herein.

Gene therapy methods and methods of delivering genes to subjects, for example, using adeno-associated viruses (AAV) and AAV vectors, are described in U.S. Pat. No. 6,967,018; Intl Pat. Pub. No. WO 2014/093622; U.S. Pat. App. Pub. No. 2008/0175845; U.S. Pat. App. Pub. No. 2014/0100265; EP 2432490; EP 2352823; EP 2384200; Intl Pat. App. Pub. No. WO 2014/127198, Intl Pat. App. Pub. No. WO 2005/122723; Intl Pat.

App. Pub. No. WO 2008/137490; Intl Pat. App. Pub. No. WO 2013/142114; Intl Pat. App. Pub. No. WO 2006/128190; la' Pat. App. Pub. No. WO 2009/134681; EP 2341068; Intl Pat. App. Pub. No. WO 2008/027084; la' Pat. App. Pub. No. WO 2009/054994; Intl Pat. App. Pub. No. WO 2014059031; U.S. Pat. No. 7,977,049; and Intl Pat. App. Pub. No. WO 2014/059029, each of which is incorporated herein by reference in its entirety.

### Subjects

Embodiments provide methods for treating a subject. In some embodiments, the subject is a human. In some embodiments, the subject is a human having or in need of a treatment for a Rasopathy (see, e.g., Rauen “The RASopathies” Annu Rev Genomics Hum Genet. 2013; 14: 355-369, incorporated herein by reference). In some embodiments, the subject is a human having or in need of a treatment for neurofibromatosis type 1. In some embodiments, the subject is a human having or in need of a treatment for a MPNST.

In some embodiments, the technology provides a method for treating a neurofibromatosis in a subject. In some embodiments, methods comprise administering to the subject an effective amount of a NF1-GRD gene therapy construct (e.g., an AAV vector as described herein comprising a nucleotide sequence encoding a NF1-GRD and/or a NF1-GRD linked to a CAAX motif-containing partial or complete Ras HVR sequence, e.g., a sequence from a Ras HVR (e.g., a partial or complete Ras HVR sequence (e.g., a sequence comprising a CAAX motif))). In some embodiments, the subject has a neurofibromatosis that is neurofibromatosis type 1, Schwannomatosis, a sporadic schwannoma, and/or a disease otherwise characterized by the presence of a tumor of Schwann cell origin. In some embodiments, the tumor of Schwann cell origin is a schwannoma or a malignant peripheral nerve sheath tumor (MPNST).

In some embodiments, the subject exhibits no detectable symptoms and/or phenotypes of disease and in some embodiments the subject exhibits detectable symptoms and/or phenotypes of disease. One serious symptom that affects a person with neurofibromatosis type 1 is a malignant peripheral nerve sheath tumor (MPNST). MPNST typically forms from unexpected growth of a preexisting neurofibroma, particularly a plexiform neurofibroma. The first symptom is typically unexplained or sudden pain in the area in or around existing tumors. Other symptoms may include, e.g., swelling (often painless) in the extremities (arms or legs), difficulty moving the extremity that has the tumor, and/or soreness localized to the area of the tumor or in the extremity. In some instances, neurofibromatosis type 1 is associated with learning disabilities in individuals affected by the disease. In some instances, the disease is associated with a partial absence seizure disorder. In some instances, neurofibromatosis type 1 is associated with poor language, visual-spatial skills, learning disability (e.g., attention deficit hyperactivity disorder), headache, epilepsy, or the like. In some embodiments, the subject is diagnosed as having a disease based on a genetic test, e.g., a genetic test indicating a genetic defect producing aberrant NF1 activity and/or aberrant Ras activity. In some embodiments, a subject has a point mutation, deletion, insertion, microdeletion, and/or splicing mutations of the neurofibromin 1 (NF1) tumor suppressor gene, e.g., at 17q11.2. Detection of such genetic markers, in various embodiments, is provided by techniques known in the art, e.g., a nucleic acid amplification-based test (e.g., PCR), a probe-based method (e.g., FISH), and/or nucleic acid sequencing.

In some embodiments, a biomarker is used for detecting disease or to classify disease (e.g., a diagnostic biomarker). In some embodiments, a biomarker is used to predict a response to a therapy or to predict an adverse event (e.g., a predictive biomarker). In some embodiments, a biomarker is used to indicate an effective drug dose (e.g., a metabolic and/or a pharmacodynamic biomarker). In some embodiments, a biomarker is used to estimate the chances of progression or recurrence (e.g., an outcome biomarker). In some embodiments, a biomarker indicates the presence of a disease (e.g., neurofibromatosis), the presence of a specific tumor type (e.g., a MPNST), the presence of a nontumor phenotype (e.g., pain), indicates a cumulative disease burden (e.g., systemic tumor burden), indicates disease progression (e.g., growth of plexiform neurofibroma), or indicates malignant transformation (e.g., MPNST formation from a neurofibroma). Several neurofibromatosis-associated biomarkers are known in the art. See, e.g., Hanemann at al. (2016) “Current status and recommendations for biomarkers and biobanking in neurofibromatosis” Neurology 87 (Supplement 1): S40-S48, incorporated herein by reference.

In some embodiments, a method is provided for treating a subject in need of such treatment with an effective amount of a nucleic acid construct as described herein (e.g., one or more doses of a therapeutically or prophylactically effective amount of a nucleic acid, nucleic acid construct, AAV vector, or pharmaceutical composition as described herein). In some embodiments, the method comprises administering to the subject an effective amount of a nucleic acid construct as described herein (e.g., one or more doses of a therapeutically or prophylactically effective amount of a nucleic acid, nucleic acid construct, AAV vector, or pharmaceutical composition as described herein) in any one of the pharmaceutical preparations described herein. The subject can be any subject in need of such treatment.

In some embodiments, a subject is tested to assess the presence, the absence, or the level of a disease (e.g., a Rasopathy, a neurofibromatosis (e.g., neurofibromatosis type 1), a cancer (e.g., a MPNST)), e.g., by assaying or measuring a biomarker, a metabolite, a physical symptom, an indication, etc., to determine the risk of or assess the presence, the absence, or the level of a disease (e.g., a Rasopathy, a neurofibromatosis (e.g., neurofibromatosis type 1), a cancer (e.g., a MPNST)), and thereafter the subject is treated with a nucleic acid, nucleic acid construct, AAV vector, or pharmaceutical composition as described herein) based on the outcome of the test. In some embodiments, a patient is tested, treated, and then tested again to monitor the response to therapy. In some embodiments, cycles of testing and treatment may occur without limitation to the pattern of testing and treating (e.g., test/treat, test/treat/test, test/treat/test/treat, test/treat/test/treat/test, test/treat/treat/test/treat/treat, etc), the periodicity, or the duration of the interval between each testing and treatment phase.

### Kits

Some embodiments relate to kits. In some embodiments, kits comprise a nucleic acid construct as described herein (e.g., one or more doses of a therapeutically or prophylactically effective amount of a nucleic acid, nucleic acid construct, AAV vector, or pharmaceutical composition as described herein). In some embodiments, kits comprise a nucleic acid construct as described herein (e.g., one or more doses of a therapeutically or prophylactically effective amount of a nucleic acid, nucleic acid construct, AAV vector, or pharmaceutical composition as described herein) and a means for intravenous, intraperitoneal, subcutaneous, and/or intramuscular administration of the nucleic acid construct. In some embodiments, kits comprise a nucleic acid construct as described herein (e.g., one or more doses of a therapeutically or prophylactically effective amount of a nucleic acid, nucleic acid construct, AAV vector, or pharmaceutical composition as described herein) and a means for intravenous administration or injection, intraperitoneal administration or injection, intramuscular administration or injection, and/or subcutaneous administration or injection.

The technology provided herein also includes kits, e.g., for use in the instant methods. Kits of the technology comprise one or more containers comprising a nucleic acid construct as described herein (e.g., one or more doses of a therapeutically or prophylactically effective amount of a nucleic acid, nucleic acid construct, AAV vector, or pharmaceutical composition as described herein). In some embodiments, kits comprise a second agent. In some embodiments, kits further comprise instructions for use in accordance with any of the methods provided herein. The kit may further comprise a description of selecting an individual for suitable treatment. Instructions supplied in the kits of the technology are typically written instructions on a label or package insert (e.g., a paper insert included with the kit), but machine-readable instructions (e.g., instructions carried on a magnetic or optical storage disk) are also contemplated. In some embodiments, the kit is a package containing a sealed container comprising any one of the preparations described above, together with instructions for use. The kit can also include a diluent container containing a pharmaceutically acceptable diluent. The kit can further comprise instructions for mixing the preparation and the diluent. The diluent can be any pharmaceutically acceptable diluent. Well-known diluents include 5% dextrose solution and physiological saline solution. The container can be an infusion bag, a sealed bottle, a vial, a vial with a septum, an ampule, an ampule with a septum, or a syringe. The containers can optionally include indicia indicating that the containers have been autoclaved or otherwise subjected to sterilization techniques. The kit can include instructions for administering the various solutions contained in the containers to subjects.

### Combination Therapies

Some embodiments provide a combination therapy, e.g., administration of a nucleic acid construct as described herein (e.g., one or more doses of a therapeutically or prophylactically effective amount of a nucleic acid, nucleic acid construct, AAV vector, or pharmaceutical composition as described herein) in combination with another (e.g., second) medical intervention (e.g., drug, surgery, radiation, etc.). In some embodiments, combination therapies comprise administration of a nucleic acid construct as described herein before a second medical intervention. In some embodiments, combination therapies comprise administration of a nucleic acid construct as described herein after a second medical intervention. In some embodiments, combination therapies comprise administration of a nucleic acid construct as described herein substantially concurrently with a second medical intervention. In some embodiments, a combination therapy comprises administering a nucleic acid construct as described herein and a farnesyltransferase inhibitor (e.g., tipifarnib) or a geranylgeranyltransferase inhibitor. In some embodiments, a combination therapy comprises administering a nucleic acid construct as described herein and an agent targeting the RAF/MAPK/ERK pathway and/or the PI3K/AKT/mTOR pathway. In some embodiments, a combination therapy comprises administering a nucleic acid construct as described herein and a BRAF inhibitor, a MEK inhibitor, an mTOR inhibitor, a c-Kit inhibitor, an EGFR inhibitor, an anti-angiogenic agent, a proteasome inhibitor, chemokine receptor inhibitor, a tyrosine kinase inhibitor, and/or a vascular endothelial growth factor (VEGF) inhibitor. In some embodiments, a combination therapy comprises administering a nucleic acid construct as described herein and one or more of sorafenib (NEXAVAR), sirolimus, everolimus (AFINITOR), imatinib mesylate (GLEEVEC), nilotinib (TASIGNA), sunitinib (SUTENT), erlotinib (TARCEVA), rapamycin, ranibizumab, selumetinib (AZD6244), PD-0325901, pirfenidone, cabozantinib (XL184), bortezomib, or bevacizumab (AVASTIN). In some embodiments, a combination therapy comprises administering a nucleic acid construct as described herein and a chemotherapeutic agent (e.g., vincristine, carboplatin, pegylated interferon-alfa-2b). In some embodiments, a combination therapy comprises administering a nucleic acid construct as described herein and a microRNA, siRNA, or antisense nucleic acid.

### Uses

In various embodiments, the technology finds use in genetic therapy. In additional embodiments, the technology finds use in research (e.g., in vitro, ex vivo, and/or in vivo) to study a Rasopapthy, neurofibromatosis type 1, and/or MPNST diseases. In some embodiments, the technology provides a nucleic acid for use in studying disease in a model system (e.g., a mammal (e.g., a mouse, rat, dog) and/or a cell culture system).

In some embodiments, the technology provides a nucleic acid for use in the preparation of a medicament. In some embodiments, the technology provides a nucleic acid for use in the preparation of a medicament for treating a subject. In some embodiments, the technology provides a nucleic acid for use in the preparation of a medicament to treat a subject having a Rasopathy, neurofibromatosis type 1, and/or a MPNST disease. In some embodiments, the technology finds use in constructing a genetic delivery vector, e.g., an AAV vector as described herein comprising a nucleic acid encoding a NF1-GRD construct and/or a nucleic acid encoding a NF1-GRD construct further comprising a CAAX motif-containing partial or complete Ras HVR sequence, e.g., a sequence from a Ras HVR (e.g., a partial or complete Ras HVR sequence (e.g., a sequence comprising a CAAX motif)).

This technology provides a recombinant construct that adds a membrane-targeting CAAX motif-containing partial or complete Ras HVR sequence to the GAP-related domain (GRD) sequence of NF1. The construct encodes an anti-Ras protein that attaches to the plasma membrane. Experimental data described below indicated that the construct enhanced the suppression of the Ras pathway, which is therapeutically targeted in NF1-related MPNSTs, neurofibromatosis in general, and a wide range of other Rasopathies. This construct finds use in adeno-associated virus in gene therapy, which provides the first causative treatment for these patients.

### Examples

During the development of embodiments of the technology provided herein, experiments were conducted to screen a panel of AAV vectors in MPNST and primary Schwann cells to measure transduction efficacies of different AAV serotypes. Further, during the development of embodiments of the technology presented herein, experiments were conducted to test the inhibition of the Ras pathway in NF1-related MPNST cells using a GRD engineered for membrane-targeting.

### Materials and Methods

Tissue culture and cell lines. NF90-8, ST88-14, sNF96.2, and STS26T malignant peripheral nerve sheath tumor (MPNST) cell lines were cultured in DMEM (ATCC) media supplemented with 10% FBS (Sigma) and penicillin/streptomycin (Gibco). Human

Schwann cells (HSC) isolated from spinal nerve were purchased from ScienCell Research Laboratories and maintained in DMEM media supplemented with 3% FBS, penicillin/streptomycin, 20 ng/ml heregulin-1 (PeproTech), and 2 μM forskolin (Sigma).

Reagents and antibodies. Rabbit anti-phospho-Erk1/2 (p44/42 MAPK) (Thr 202/Tyr 204) antibody (#9101) and anti-Erk1/2 (p44/42 MAPK) antibody (#9102) were purchased from Cell Signaling Technologies. Rabbit anti-NF1 antibody (A300-140A) was purchased from Bethyl laboratories, mouse anti-HA antibody (26183) was purchased from Invitrogen, and anti-beta-actin (C-11) HRP antibody was purchased from Santa Cruz Biotech. Active Ras Detection Kit (#8821) was purchased from Cell Signaling Technology. NucBlue Live Ready Probes was purchased from Invitrogen. NF1-GRD constructs. Human NF1 transcript variant 2 (NCBI Accession NM_000267.3, SEQ ID NO: 1), which encodes a protein of 2818 amino acids (NCBI Accession NP_000258.1, SEQ ID NO: 2), was used as the template sequence. The portion of the NF1 transcript sequence encoding the amino acid sequence of the GRD region of NF1 (e.g., amino acids 1172-1538 (see reference 39)) was cloned with a C-terminal 2× HA tag into the lentivirus pFUGW and pscAAV-MCS (Cell Biolabs, VPK-430) vectors. In some embodiments, the GRD region of NF1 is provided by amino acids 1187-1536 or 1198-1530 of SEQ ID NO: 2 (see NCBI Reference Sequence: NP_000258.1). To create a membrane-targeting GRD construct (“GRD-C10”), a 2× HA sequence was fused to the N-terminus of GRD and a sequence encoding the H-Ras C-terminal 10 amino acids (GCMSCKCVLS, SEQ ID NO: 3) containing the CAAX motif-containing partial HRas HVR sequence was attached in frame to the GRD C-terminus.

Lentiviral production and transduction. EGFP or GRD-2HA was cloned in pFUGW vector and the plasmid was transfected with CMVAR8.91 and pMD.G in 293T cells by Lipofectamine 2000 (Invitrogen). Virus was harvested after 48 hours and used to infect MPNST cells by incubating with 8 μg/ml polybrene (Sigma).

AAV plasmids. Thirteen hybrid pAAV-Rep-Cap (pAAV-RC) vectors were obtained that encode the rep of AAV2 and variable cap genes of different serotypes. Among them, pAAV2-RC was purchased from Clontech; pAAV-RC3B, pAAV-RC4, pAAV-RC6, and pAAV-DJ were purchased from Cell Biolabs; and pAAV2/1, pAAV2/5 JC, pAAV2/7, pAAV2/8, pAAV9n, pAAV2/rh10, pAAV2/hu11, and pAAV2/rh32.33 were obtained from Penn Vector Core of the University of Pennsylvania. For AAV packaging, the pHelper plasmid and AAVpro-293T cells from Clontech were used.

AAV production and purification. pAAV-Rep-Cap, pscAAV, and pHelper plasmids in equal amounts were transfected in AAVpro-293T cells by Lipofectamine 2000 in DMEM media supplemented with 10% FBS. After 3 days, cells were harvested and AAVs were purified by AAVpro Purification Kit of Clontech following manufacturer'instructions. Viral titers were determined by AAVpro Titration Kit from Clontech using real-time PCR.

AAV transduction and fluorescence microscopy. Five thousand HSC or MPNST cells were plated in each well of a flat-bottomed 96-well plate. AAV vectors comprising coding sequence for enhanced GFP (EGFP) were added to the cells at MOI 1000 or MOI 5000 in 60 μl of regular growth media and incubated for 5 days. After the 5-day growth, the growth medium was changed to FreeStyle 293 Expression medium (Gibco) without Phenol Red. NucBlue Live Cell Stain ReadyProbes (Invitrogen) was added to stain nuclei with blue fluorescence. The cells were grown to confluence and the fluorescence signals were quantified on a PerkinElmer VICTOR3 1420 Multilabel Counter with a green fluorescence (485/535 nm) filter set. EGFP signals were subsequently examined on an immunofluorescence microscope.

Immunofluorescence Staining. The staining followed previously published procedures [40]. Cells were grown in medium on chamber slides (Nunc) and treated as described previously. Cells were washed with PBS and fixed for 10 minutes with 4% paraformaldehyde solution and permeated with methanol for 2 minutes with 3 washes in PBS in between each step and afterward. The slides were first blocked by 10% goat serum in PBS for 1 hour at room temperature, incubated with the first antibody, and subsequently incubated with AlexaFluor 488 (Green) secondary antibody (Invitrogen) in 10% goat serum in PBS at room temperature. Slides were washed 3 times in PBS in between each step and afterward. After staining, the slides were covered with mounting medium containing DAPI (Vector Laboratories) and examined on a fluorescence microscope.

Western Blots (immunoblots). Cells were lysed in buffer as described previously [41, 40]. Briefly, cells were suspended in lysis buffer (10 mM Tris-HCl (pH 7.5), 130 mM NaCl, 5 mM EDTA, 0.5% Triton X-100, 20 mM sodium phosphate (pH 7.5), 10 mM sodium pyrophosphate (pH 7.0), 50 mM NaF, 1 mM sodium orthovanadate,1 mM phenylmethylsulfonyl fluoride, and protease inhibitor cocktail (Roche)) and kept on ice for 10 minutes before being centrifuged. Cell lysates were heated for 5 minutes with LDS Sample Buffer (Invitrogen) supplemented with 100 mM DTT before loading onto a 4-12% NuPAGE Bis-Tris Gel (Invitrogen). After transfer to a PVDF membrane (Bio-Rad), immunostaining was performed according to standard procedures. Signals were visualized using the Super Signal chemiluminescent system (Pierce, Rockford, Ill.).

Assay of active Ras. Ras activities in MPNST cells were examined by the Active Ras Detection Kit (#8821, Cell Signaling Technology) following manufacturer instructions. Briefly, the cell lysates were incubated with GST-Rafl-RBD and glutathione resin. After wash and centrifugation, the bound fractions of Ras were dissociated and denatured in SDS sample buffer with DTT and examined by anti-Ras western blotting. Pre-incubating the lysates with 0.1 mM GTP_{Y}S or 1 mM GDP before binding with GST-Rafl-RBD provided the positive and negative controls, respectively.

Cells Growth Assay. The viable cells were measured with Cell Counting Kit-8 (Dojindo Laboratories, Japan) containing WST-8 tetrazolium salt at 450 nm on a PerkinElmer VICTOR3 plate reader.

Statistical Analysis. The results are presented as a mean value plus or minus the standard deviation. Data were analyzed by GraphPad Prism 5.0. The p-values were determined by a Mantel-Cox test. A p-value under 0.05 was accepted as statistically significant.

### Example 1—NF1-GRD Suppresses Ras Activity and Growth of MPNST Cells

During the development of embodiments of the technology, it was contemplated that expressing the GRD domain of NF1 (NF1-GRD) could restore normal growth in NF1−/− Nf1-deficient hematopoietic cells, fibroblasts, and neural stem cells [16, 17]. Further, it was contemplated that NF1-GRD could restore normal growth of MPNST cells [18, 19].

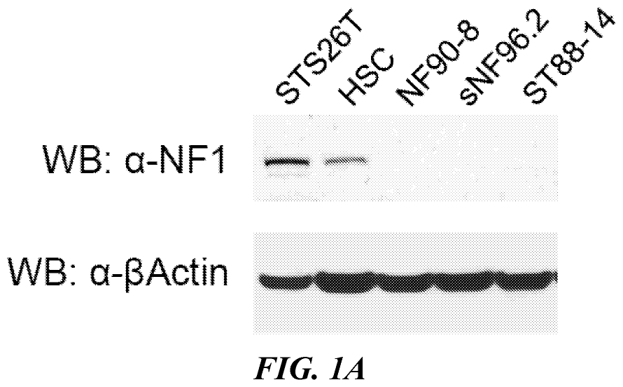

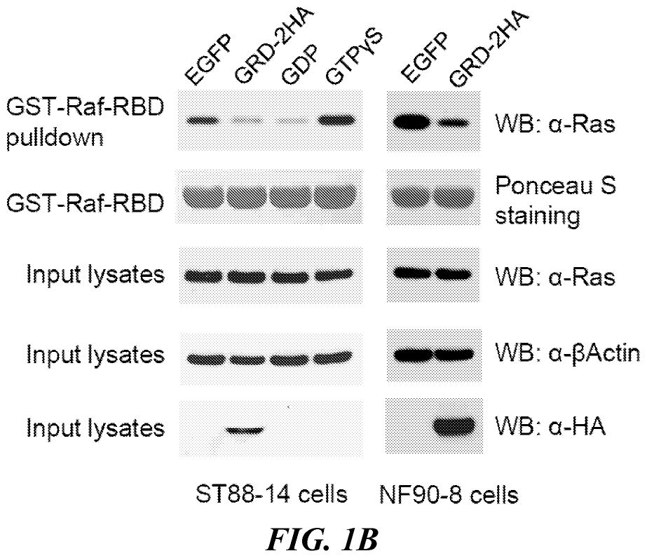

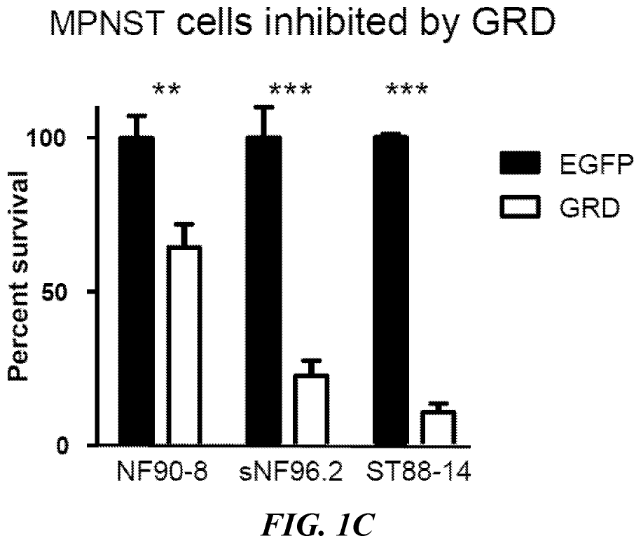

Accordingly, during the development of embodiments of the technology provided herein, experiments were conducted in which the GRD domain of human NF1 was subcloned into the pFUGW lentivirus (LV) vector with a C-terminal double hemagglutinin (HA) tag (2HA) for sensitive in vitro and in vivo detection. The human MPNST cell lines NF90.8, sNF96.2, and ST88-14 lacking wildtype (WT) NF1 gene and STS26T cells comprising a functional NF1 were derived from neurofibromatosis 1 patients [20] and primary human Schwann cells were isolated from spinal nerve. Western blots were used to confirm NF1 expression in lysates prepared from the MPNST cells (FIG. 1A). Further, the GRD-2HA construct significantly reduced aberrant Ras activity in ST88-14 and NF90.8 cells in the GSTRafl-RBD pulldown assay (FIG. 1B), indicating that the GAP domain of NF1 complements the inability of these cells to inactivate Ras. In particular, the data collected indicated that the introduction of the NF1-GRD recombinant construct into the MPNST cells via lentiviral transduction significantly suppressed the growth of NF90.8, sNF96.2, and ST88-14 cells compared to the EGFP control construct (FIG. 1C).

### Example 2—Testing Transduction Efficiency of AAV Vectors

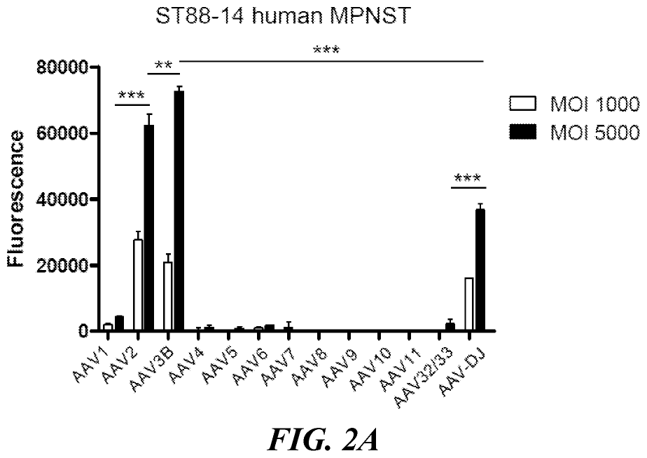

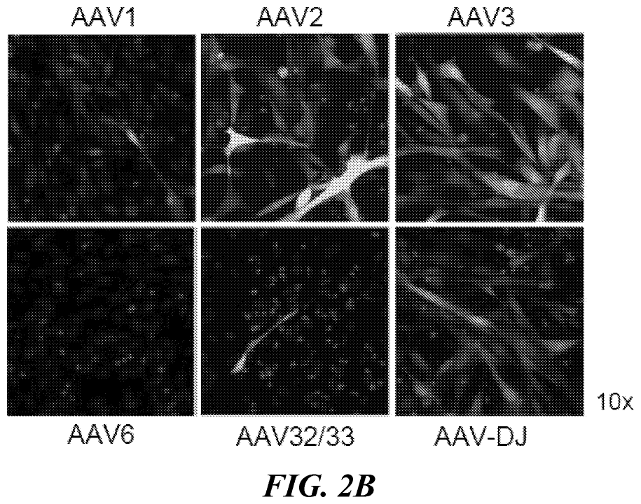

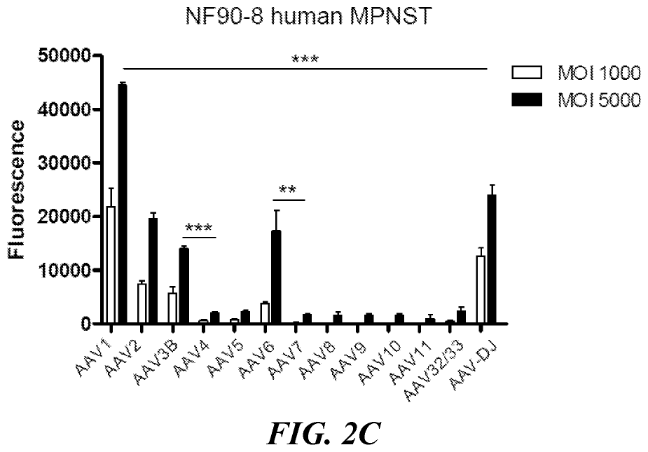

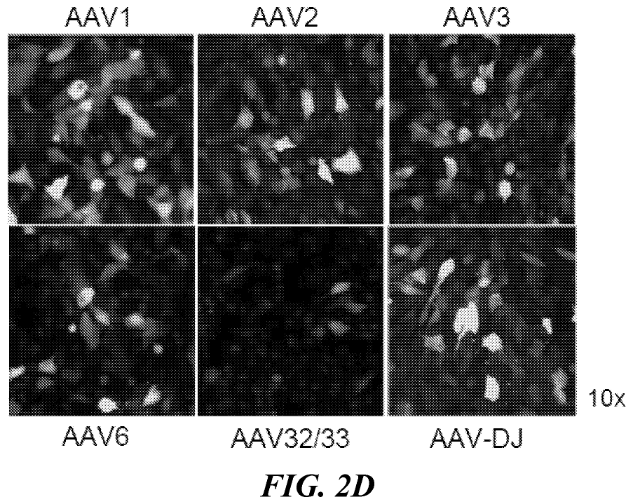

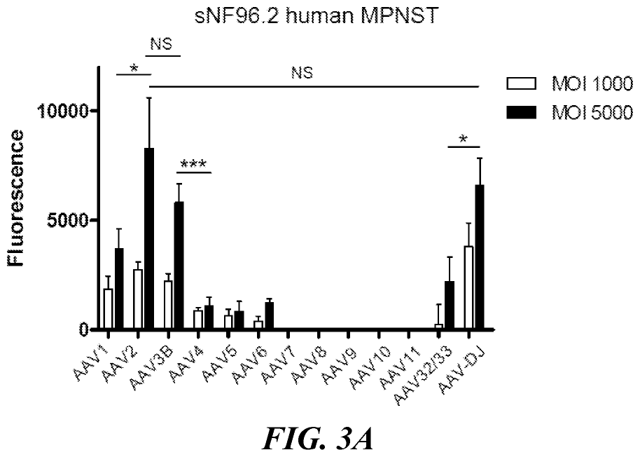

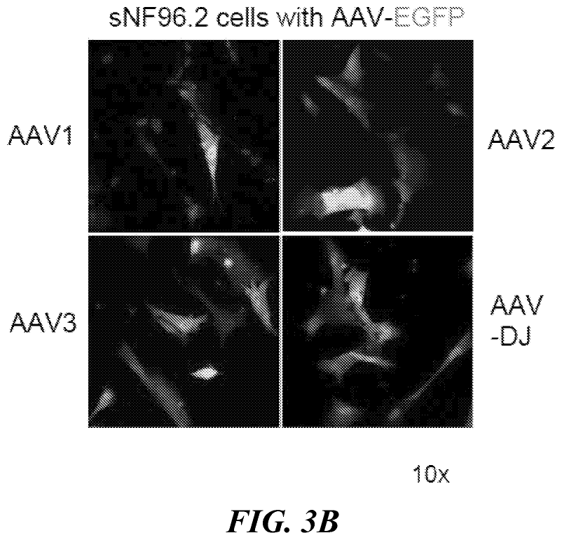

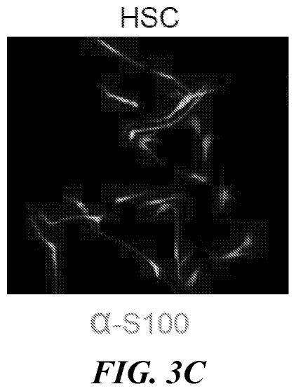

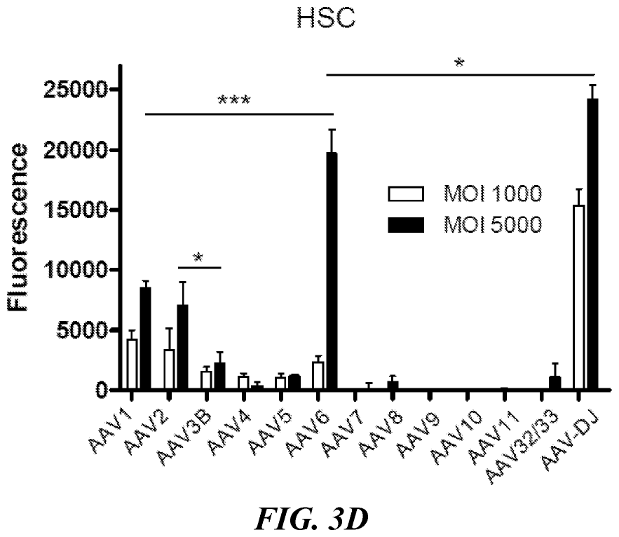

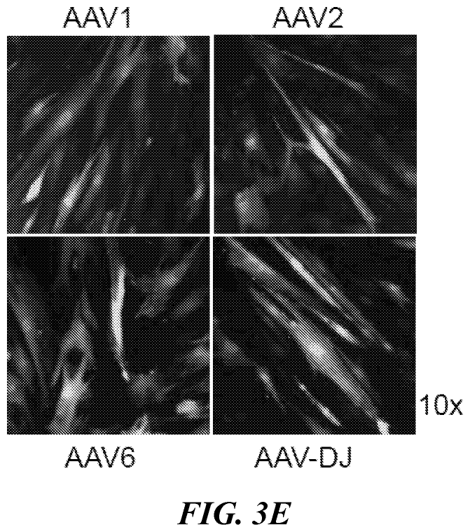

Next, during the development of embodiments of the technology provided herein, experiments were conducted to compare the transduction efficacy of a panel of commercially available AAV vectors in MPNST and primary human Schwann cells. In particular, enhanced green fluorescent protein (EGFP) was cloned into each AAV vector, introduced into cells, and EGFP expression was quantified by measuring the green fluorescent signals. In these experiments, thirteen hybrid pAAV-Rep-Cap (pAAV-RC) vectors, which encode the rep of AAV2 and variable cap genes of different serotypes, were compared. These vectors were AAV1 (pAAV2/1), AAV2 (pAAV2-RC), AAV3B (pAAV-RC3B), AAV4 (pAAV-RC4), AAV5 (pAAV2/5 JC), AAV6 (pAAV-RC6), AAV7 (pAAV2/7), AAV8 (pAAV2/8), AAV9 (pAAV9n), AAV10 (pAAV2/rh10), AAV11 (pAAV2/hu11), AAV32/33 (pAAV2/rh32.33), and the synthetic AAV-DJ (pAAVDJ) [21-25], along with the pHelper and self-complementary pscAAV (pscAAV-MCS) expression vector to enhance transduction [26]. The pscAAV vector uses the CMV promoter for the transgene. After production, purification, and quantification of AAVs, 5000 ST88-14, NF90-8, or sNF96.2 cells were plated in 96-well plates and incubated with each individual AAV at MOI 1000 or 5000. Cells were grown to confluence (e.g., approximately five days) as confirmed optically by microscope. Green fluorescent signals in each well were quantified by a plate reader to measure the expression levels of EGFP in the transduced cells. The data collected indicated that the vectors AAV2, AAV3B, and AAV-DJ provided superior transduction of EGFP in ST88-14 cells at MOI 5000 (FIG. 2A). The data collected also indicated that the vectors AAV1, AAV2, AAV3B, AAV6, and AAV-DJ provided significant transduction at MOI 5000 in contrast to the other AAVs (FIG. 2C). Data collected in experiments performed at a MOI 1000 followed a similar trend (FIG. 2A and FIG. 2C). The results were consistent with the observation of transduced cells by fluorescence microscopy (FIG. 2B and FIG. 2D). In sNF96.2 cells, the data indicated that cells transduced with the vectors AAV1, AAV2, AAV3B, and AAV-DJ again provided the highest fluorescent signals at both MOI 5000 and 1000 (FIG. 3A and FIG. 3B).

### Example 3—Transduction of Human Schwann Cells

It has been established that neurofibromastosis 1 and its malignant form MPNST originate from Schwann cells [2, 27]. Accordingly, during the development of embodiments of the technology provided herein, it was contemplated that gene replacement therapy restoring NF1 functions in NF1 haploid cells (e.g., Schwann cells) before malignant transformation would benefit patients. Thus, during the development of embodiments of the technology provided herein, experiments were conducted to transduce primary human Schwann cells (HSC) that are positive for S-100 (FIG. 3C) (a protein that is found in cells derived from the neural crest) with the panel of thirteen AAV vectors encoding EGFP (tested above). The data collected from the experiments indicated that the vectors AAV 1, AAV2, AAV6, and AAV-DJ delivered EGFP most efficaciously (FIG. 3D and FIG. 3E).

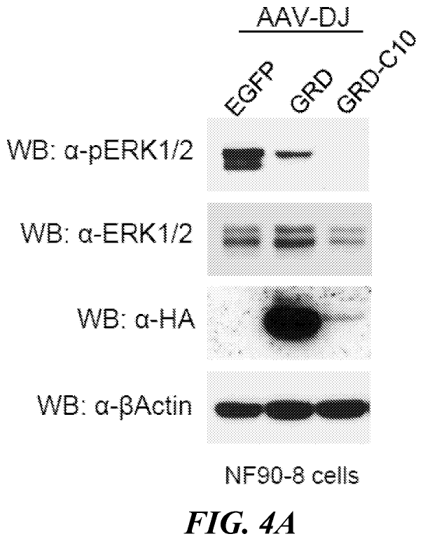

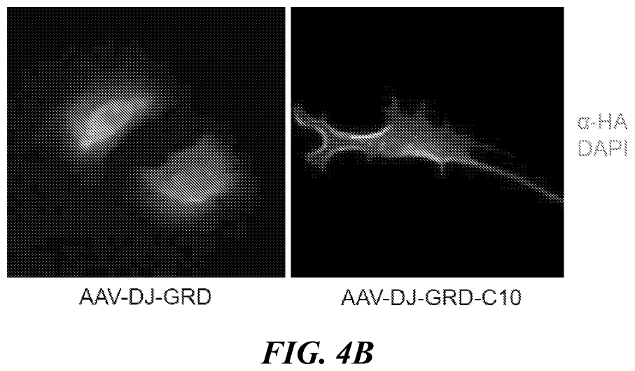

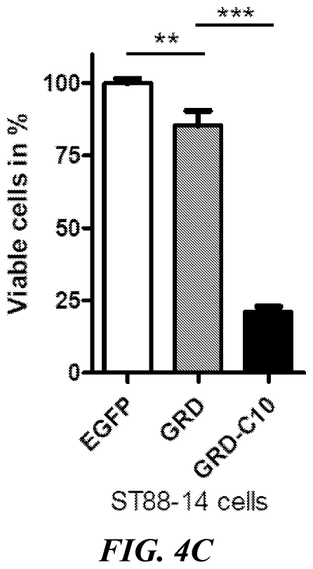

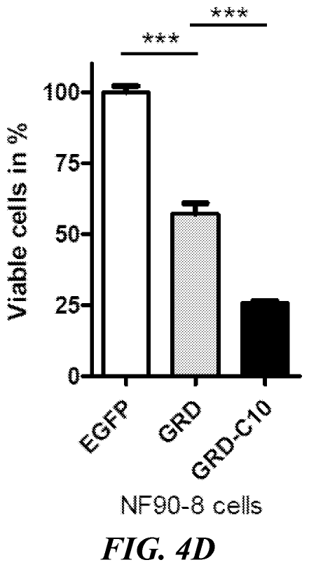

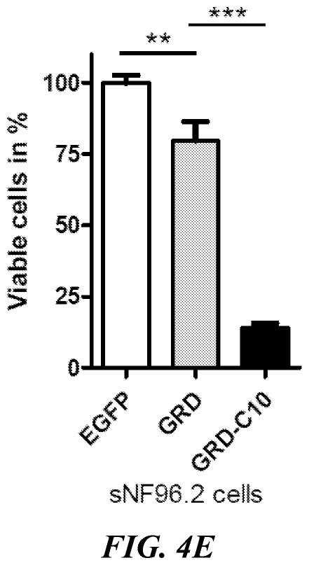

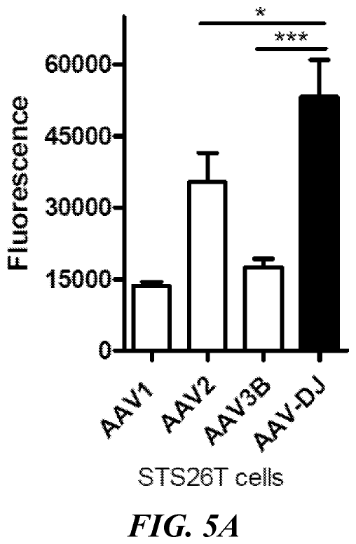

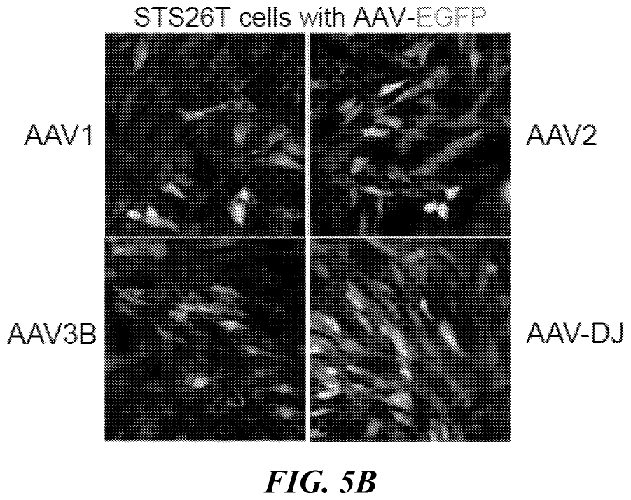

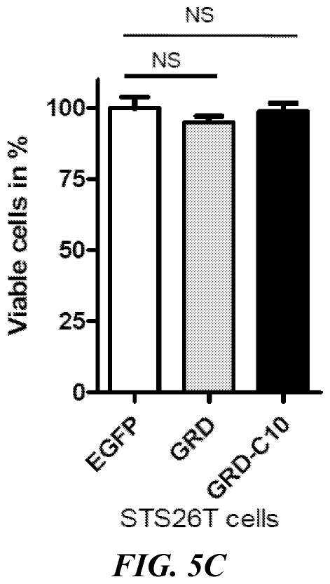

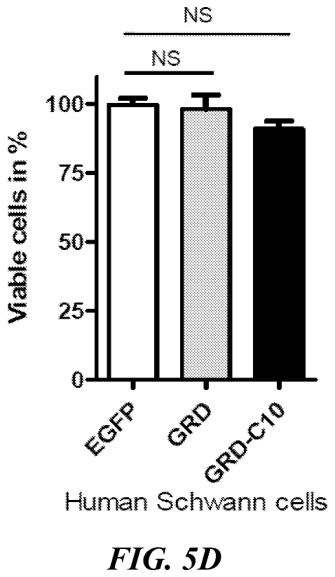

Next, experiments were conducted to test NF1-GRD constructs cloned into AAV-DJ, one of the AAV vectors that the data indicated to be consistently effective for transducing MPNST cells and HSC. Ras proteins are attached to the cellular membrane through prenylation via a CAAX motif and palmitoylation of cysteine residues or polybasic residues located in the C-terminal hypervariable region (HVR) that is sufficient to confer targeting to plasma membrane [28, 29]. Accordingly, during the development of embodiments of the technology provided herein, experiments were conducted to construct NF1-GRD fused at its C-terminus to the 10 amino acids of the H-Ras C-terminus (C10) comprising the CAAX motif with partial HRas HVR and further comprising a double HA tag at the N-terminus. When transduced by AAV-DJ into NF90-8 cells, GRD-C10 drastically outperformed GRD in suppressing the phosphorylation of Erk1/2 (pErk1/2). In particular, while a reduced pERK1/2 signal was observed in cells transduced with GRD, no pErk1/2 signal was observed in cells transduced with GRD-C10 (FIG. 4A). Further, pErk1/2 was not detected even though GRD-C10 was expressed at a relatively low level (FIG. 4A). Immunofluorescence staining of HA confirmed the targeting of GRD-C10 to the membrane (FIG. 4B). The data indicated that GRD-C10 markedly outperformed GRD in suppressing the growth of ST88-14, NF90-8, and sNF96.2 cells (FIG. 4C, FIG. 4D, and FIG. 4E). In contrast, despite being transducible with AAV-DJ (FIG. 5A and FIG. 5B), STS26C cells, a spontaneous MPNST cell line with intact NF1 (FIG. 1A), were not affected by either GRD or GRD-C10 transduced by AAV-DJ (FIG. 5C). Similarly, growth of HSCs was not suppressed by GRD and not by GRD-C10 at a significant level (FIG. 5D).

### Example 4—Comparing Ras HVR Sequences

As shown in Example 3, fusing the C-terminal 10 amino acids of H-Ras (comprising the CAAX motif and partial HVR including two palmitoylation-targeted cysteine residues) to GRD (GRD-C10) enhanced membrane-targeting and Ras inhibition of GRD. Thus, during the development of embodiments of the technology provided herein, experiments were conducted to test multiple other CAAX-containing partial or complete Ras HVR sequences for membrane targeting and Ras inhibition activities. In particular, experiments were conducted to test and compare the membrane-targeting and Ras-targeting efficiencies of C-terminal sequences from the hypervariable regions (HVR) of the Ras proteins HRas, NRas, KRas4A, and KRas4B.

Each C-terminal HVR, or a portion thereof, was fused with NF1-GRD tagged with an N-terminal 2× HA sequence (“2HA-GRD”) as described herein (see, e.g., Materials and Methods, supra). The complete HRas, NRas, KRas4A, and KRas4B HVR amino acid sequences are shown in FIG. 6A. The HRas, NRas, and KRas4A HVR amino acid sequences are amino acids 165-189 of the HRas, NRas, and KRas4A amino acid sequences, respectively. The KRas4B amino amino acid sequence is amino acids 165-188 of the KRas4B amino acid sequence.

HRaS HVR
(SEQ ID NO: 10)
HKLRKLNPPDESGPGCMSCKCVLS
NRaS HVR
(SEQ ID NO: 11)
YRMKKLNSSDDGTQGCMGLPCVVM
KRaS4A HVR
(SEQ ID NO: 12)
YRLKKISKEEKTPGCVKIKKCIIM
KRas4B HVR
(SEQ ID NO: 13)
KHKEKMSKDGKKKKKKSKTKCVIM

The NF1-GRD amino acid sequence is provided by amino acids 1172-1538 of the human NF1 provided by Genbank Accession NM_000267.3, incorporated herein by reference. The constructs comprised the terminal 10, 21, 22, or 24 amino acids from the C-termini of the Ras HVR sequences and thus each included the C-terminal CAAX motif sequence and palmitoylation-targeted cysteine residues and/or polybasic amino acid sequences. Constructs comprising 10, 21, 22, or 24 amino acids from the C-termini of the Ras HVR sequences are denoted as “C10”, “C21”, “C22”, and “C24” constructs, respectively. The C24 constructs comprised full HVR sequences. GRD without a Ras HVR sequence was used as a control. Thus, the GRD-HRas-C10 construct comprised the 2HA-GRD fused to the C-terminal 10 amino acids of the HRas HVR; the GRD-KRas4B-C21 construct comprised the 2HA-GRD fused to the C-terminal 21 amino acids of the KRas4B HVR; the GRD-KRas4A-C22 construct comprised the 2HA-GRD fused to the C-terminal 22 amino acids of the KRas4A HVR; the GRD-HRas-C24 construct comprised the 2HA-GRD fused to the C-terminal 24 amino acids of the HRas HVR; the GRD-NRas-C24 construct comprised the 2HA-GRD fused to the C-terminal 24 amino acids of the NRas HVR; the GRD-KRas4A-C24 construct comprised the 2HA-GRD fused to the C-terminal 24 amino acids of the KRas4A HVR; and the GRD-KRas4B-C24 construct comprised the 2HA-GRD fused to the C-terminal 24 amino acids of the KRas4B HVR.

HRas-C10
(SEQ ID NO: 14)
GCMSCKCVLS
KRas4B-C21
(SEQ ID NO: 15)
EKMSKDGKKKKKKSKTKCVIM
KRas4A-C22
(SEQ ID NO: 16)
LKKISKEEKTPGCVKIKKCIIM
HRas-C24
(SEQ ID NO: 17)
HKLRKLNPPDESGPGCMSCKCVLS
NRas-C24
(SEQ ID NO: 18)
YRMKKLNSSDDGTQGCMGLPCVVM
KRas4A-C24
(SEQ ID NO: 19)
YRLKKISKEEKTPGCVKIKKCIIM
KRas4B-C24
(SEQ ID NO: 20)
KHKEKMSKDGKKKKKKSKTKCVIM

These constructs were packaged in AAV-DJ and tested in growth inhibition assays using ipn02.3-2λ, ipNF9511bc, and ST8814 cells as described below. The ipn02.3-2λ cell line is a normal human Schwann cell line immortalized by hTert and mCdk4 and has normal NF1 expression [42]. ipNF9511bc is a plexiform NF-1 patient-derived cell line immortalized by hTert and mCdk4 and does not express NF1 [42]. ST8814 is a human MPNST cell line with no detectable NF1 expression. NF1 expression in these cell lines as reported was confirmed by immunoblot using an antibody specific for NF1 (see, e.g., FIG. 6B). As shown by FIG. 6B, ipn02.3-2λ expresses NF1 and both ipNF9511bc and ST8814 do not express NF1.

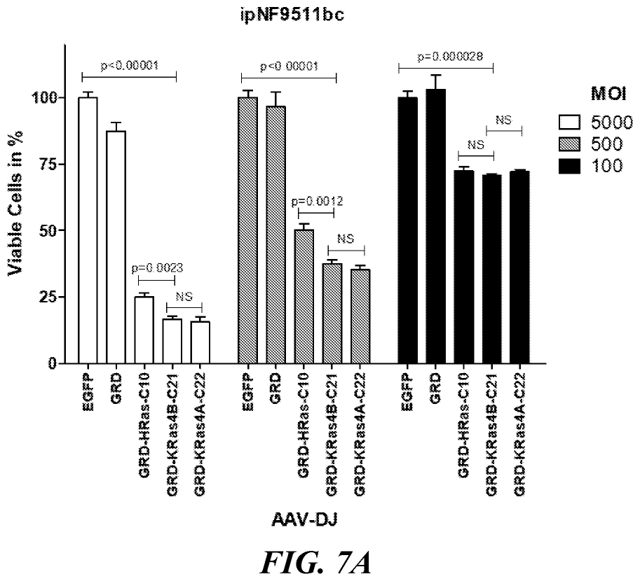

Two thousand ipNF9511bc cells were plated in one well of a 96-well plate in DMEM media supplemented with 2% FBS and antibiotics. GRD constructs comprising 2HA-GRD fused with the terminal 10, 21, or 22 amino acids from the C-termini of the Ras HVR sequences were packaged in AAV-DJ for transduction into the cells at multiplicities of infection (MOIs, virus vs target ratios) of 5000, 500, and 100. AAV-DJ-EGFP was used as a control. After 72 hours, viable cells were measured (Cell Counting Kit-8, Dojindo) and the percentage of viable cells transduced by the GRD construct was calculated as the percentage of viable control cells. Two-tailed t tests were performed by GraphPad 5.0 and p<0.05 was regarded as significant. See FIG. 7A.

Two thousand ST8814 cells were plated in one well of a 96-well plate in DMEM media supplemented with 2% FBS and antibiotics. GRD constructs comprising 2HA-GRD fused with the terminal 10, 21, or 22 amino acids from the C-termini of the Ras HVR sequences were packaged in AAV-DJ for transduction into the cells at a MOI of 500. AAV-DJ-EGFP was used as a control. After 72 hours, viable cells were measured (Cell Counting Kit-8, Dojindo) and the percentage of viable cells transduced by the GRD construct was calculated as the percentage of viable control cells. Two-tailed t tests were performed by GraphPad 5.0 and p<0.05 was regarded as significant. See FIG. 7B.

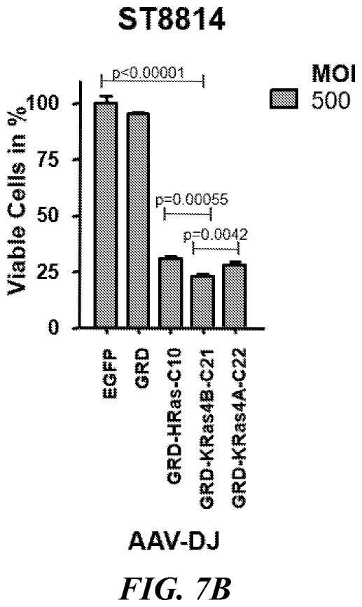

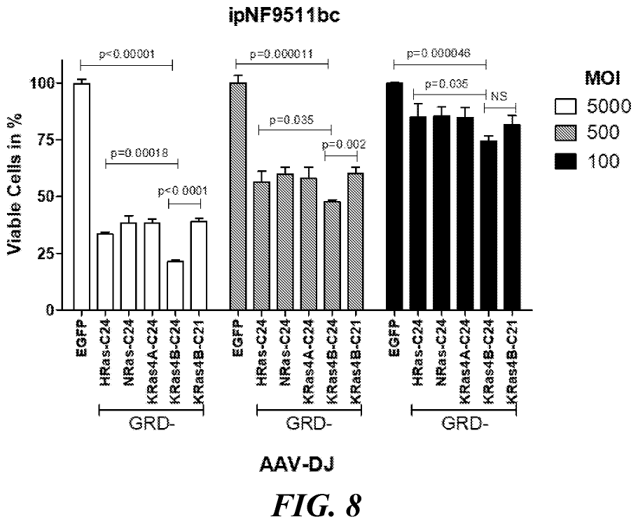

Two thousand ipNF9511bc cells were plated in one well of a 96-well plate in DMEM media supplemented with 2% FBS and antibiotics. GRD constructs comprising 2HA-GRD fused with the terminal 21 or 24 amino acids from the C-termini of the Ras HVR sequences were packaged in AAV-DJ for transduction into the cells at multiplicities of infection (MOIs, virus vs target ratios) of 5000, 500, and 100. AAV-DJ-EGFP was used as a control. After 72 hours, viable cells were measured (Cell Counting Kit-8, Dojindo) and the percentage of viable cells transduced by the GRD construct was calculated as the percentage of viable control cells. Two-tailed t tests were performed by GraphPad 5.0 and p<0.05 was regarded as significant. See FIG. 8.

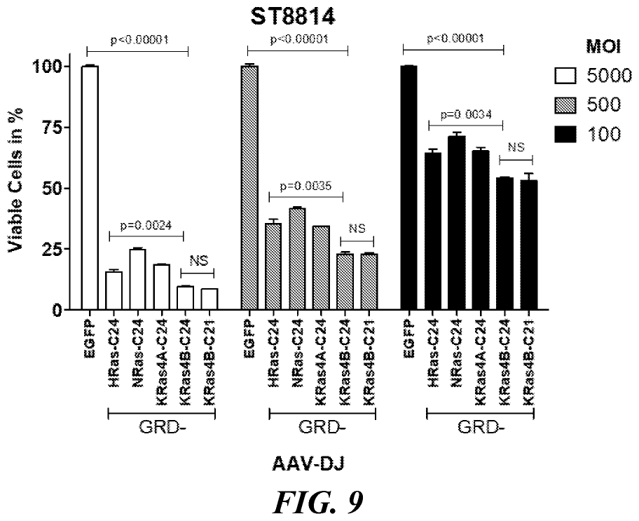

Two thousand ST8814 cells were plated in one well of a 96-well plate in DMEM media supplemented with 2% FBS and antibiotics. GRD constructs comprising 2HA-GRD fused with the terminal 21 or 24 amino acids from the C-termini of the Ras HVR sequences were packaged in AAV-DJ for transduction into the cells at multiplicities of infection (MOIs, virus vs target ratios) of 5000, 500, and 100. AAV-DJ-EGFP was used as a control. After 72 hours, viable cells were measured (Cell Counting Kit-8, Dojindo) and the percentage of viable cells transduced by the GRD construct was calculated as the percentage of viable control cells. Two-tailed t tests were performed by GraphPad 5.0 and p<0.05 was regarded as significant. See FIG. 9.

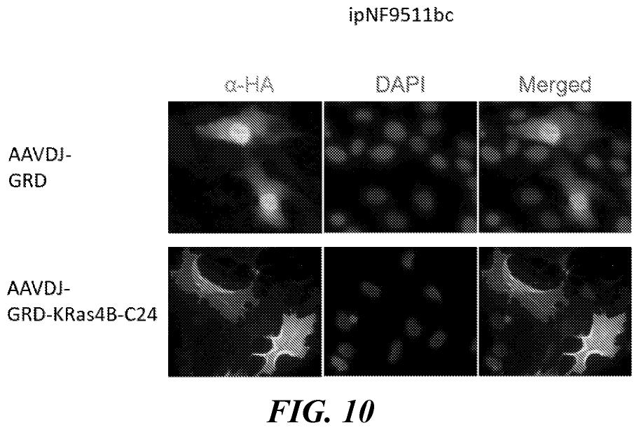

ipNF9511bc cells were transduced by AAV-DJ-GRD or AAV-DJ-GRD-KRas4B-C24 at a MOI of 5000. Both the GRD and GRD-KRas4B-C24 constructed comprised an N-terminal 2× HA tag. After 36 ours, anti-HA immunofluorescence staining was performed with Alexa488 (green) secondary antibody; DAPI was used to stain the nuclei. The images showed that GRD was localized in the cell cytosol and nuclei and that the GRD-KRas4B-C24 was localized to the cellular membrane. See FIG. 10.

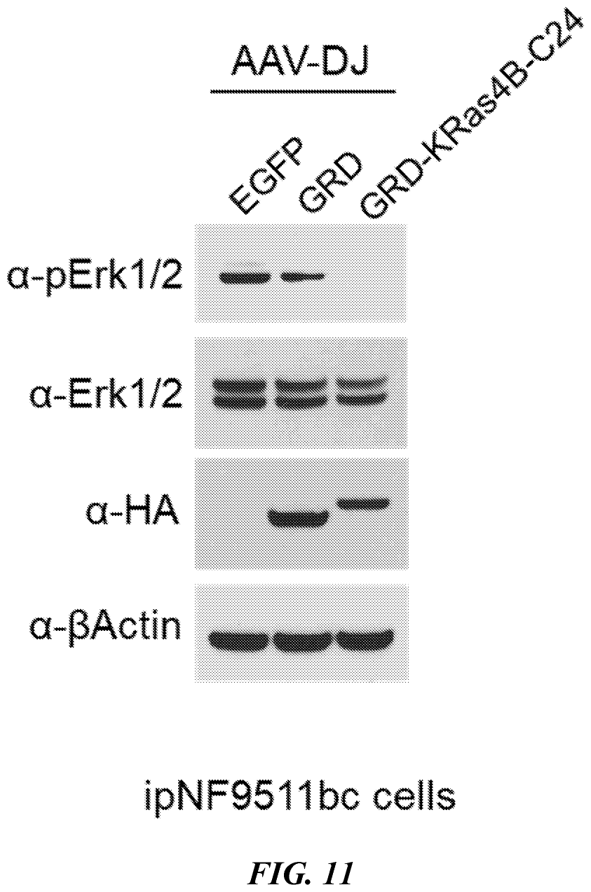

ipNF9511bc cells were transduced by AAV-DJ-GRD or AAV-DJ-GRD-KRas4B-C24 at MOI 5000 and incubated for 24 hrs. Cells were lysed and subject to Western blotting with anti-phospho-Erk1/2 (pErk1/2, Thr202/Tyr204) and anti-Erk1/2 antibodies. The Western blotting image showed that GRD-KRas4B-C24 completely inhibited Erk1/2 (p42/44) phosphorylation, in a manner far more effective than that achieved by GRD. See FIG. 11.

Data collected during these experiments indicated that GRD fusion constructs comprising an amino acid sequence from Ras HVR provided inhibition of NF1 cells. For example, GRD fusions comprising the C-terminal 24 and 21 amino acids of KRas4B HVR demonstrated highly potent inhibition of NF1 cells. The data indicated that the C-terminal 24 and 21 amino acids of KRas4B HVR provided inhibition of NF1 cells that was greater than provide by the C-terminal 24 or 10 amino acids of HRas, the 24 C-terminal amino acids of NRas, and the C-terminal 22 or 24 amino acids of KRas4A. Thus, embodiments provide GRD fusion constructs with varying inhibition of NF1 cells. In some embodiments, the technology comprises use of amino acid sequences provided by the KRas4B HVR to provide an effective NF1 gene replacement therapy by AAV.

The data showed that GRD-KRas4B-C21 and GRD-KRas4A-C22 provided significantly superior inhibition in comparison to GRD alone and GRD-HRas-C10 (GRD-C10) constructs in both ipNF9511bc and ST8814 cell lines (FIG. 7A). GRD-KRas4B-C21 performed better than GRD-KRas4A-C22 in ST8814 cells at MOI 500 with statistical significance (FIG. 2B). Furthermore, experiments compared the GRD fusion proteins with the C-terminal 24 amino acids of HRas, NRas, KRas4A, and KRas4B, which are full length or close to full length HVRs. The data indicated that GRD-KRas4B-C24 provided the most potent inhibition of the proliferation of both ipNF9511bc and ST8814 cells (FIGS. 8 and 9). Comparing GRD-KRas4B-C24 to the slightly shorter GRD-KRas4B-C21, GRD-KRas4B-C24 showed comparable potency in inhibiting ST8814 cells in all MOIs (FIG. 9) and significantly more inhibition with ipNF9511bc cells at MOI 5000 and 500 (FIG. 8). Immunofluorescence staining of GRD-KRas4B-C24 expressed in ipNF9511bc cells via AAV-DJ indicated a clear pattern of targeting to the cellular membrane, in contrast to the distribution of GRD mainly in the cytosol and nuclei (FIG. 5). The technology provides the NF1-GRD domain is various lengths. While experiments were conducted during the development of embodiments of the technology provided herein using amino acids 1172-1538 from the human NF1 (full length 2818 AAs, Genbank NM_000267.3), the technology comprises use of various shorter versions of GRD. In some embodiments, amino acids 1198-1530 (NF1-333), which provide the core domain of NF1-GRD (amino acids 1248-1477) [35], find use in the present technology and provide the same and/or similar effect.

### REFERENCES

1. Carey J C, Baty B J, Johnson J P, Morrison T, Skolnick M, Kivlin J. The genetic aspects of neurofibromatosis. Ann N Y Acad Sci. 1986; 486: 45-56.

### 2. Staedtke V, Bai R Y, Blakeley J O. Cancer of the Peripheral Nerve in Neurofibromatosis Type 1. Neurotherapeutics. 2017; 14: 298-306.

3. Abramowicz A, Gos M. Neurofibromin in neurofibromatosis type 1-mutations in

NF1gene as a cause of disease. Dev Period Med. 2014; 18: 297-306.

4. Ratner N, Miller S J. A RASopathy gene commonly mutated in cancer: the neurofibromatosis type 1 tumour suppressor. Nat Rev Cancer. 2015; 15: 290-301.
5. Gutmann D H, Aylsworth A, Carey J C, Korf B, Marks J, Pyeritz R E, Rubenstein A, Viskochil D. The diagnostic evaluation and multidisciplinary management of neurofibromatosis 1 and neurofibromatosis 2. JAMA. 1997; 278: 51-7.
6. Mautner V F, Asuagbor F A, Dombi E, Funsterer C, Kluwe L, Wenzel R, Widemann B C, Friedman J M. Assessment of benign tumor burden by whole-body MRI in patients with neurofibromatosis 1. Neuro Oncol. 2008; 10: 593-8.
7. Rodriguez F J, Folpe A L, Giannini C, Perry A. Pathology of peripheral nerve sheath tumors: diagnostic overview and update on selected diagnostic problems. Acta Neuropathol. 2012; 123: 295-319.
8. Zou C, Smith K D, Liu J, Lahat G, Myers S, Wang W L, Zhang W, McCutcheon I E, Slopis J M, Lazar A J, Pollock R E, Lev D. Clinical, pathological, and molecular variables predictive of malignant peripheral nerve sheath tumor outcome. Ann Surg. 2009; 249: 1014-22.
9. De Raedt T, Beert E, Pasmant E, Luscan A, Brems H, Ortonne N, Helin K, Hornick J L, Mautner V, Kehrer-Sawatzki H, Clapp W, Bradner J, Vidaud M, et al. PRC2 loss amplifies Ras-driven transcription and confers sensitivity to BRD4-based therapies. Nature. 2014; 514: 247-51.
10. Evans D G, Baser M E, McGaughran J, Sharif S, Howard E, Moran A. Malignant peripheral nerve sheath tumours in neurofibromatosis 1. J Med Genet. 2002; 39: 311-4.
11. Widemann B, et al. SARC006: Phase II trial of chemotherapy in sporadic and neurofibromatosis type 1 (NF1)- associated high-grade malignant peripheral nerve sheath tumors (MPNSTs). J Clin Oncol 2013; 31.
12. Jessen W J, Miller S J, Jousma E, Wu J, Rizvi T A, Brundage M E, Eaves D, Widemann B, Kim M O, Dombi E, Sabo J, Hardiman Dudley A, Niwa-Kawakita M, et al. MEK inhibition exhibits efficacy in human and mouse neurofibromatosis tumors. J Clin Invest. 2013; 123: 340-7.
13. Lisowski L, Tay S S, Alexander I E. Adeno-associated virus serotypes for gene therapeutics. Curr Opin Pharmacol. 2015; 24: 59-67.
14. Kotterman M A, Schaffer D V. Engineering adeno-associated viruses for clinical gene therapy. Nat Rev Genet. 2014; 15: 445-51.

15. Naldini L. Gene therapy returns to centre stage. Nature. 2015; 526: 351-60.

16. Hiatt K K, Ingram D A, Zhang Y, Bollag G, Clapp D W. Neurofibromin GTPase activating protein-related domains restore normal growth in Nf1−/− cells. J Biol Chem. 2001; 276: 7240-5.

17. Dasgupta B, Gutmann D H. Neurofibromin regulates neural stem cell proliferation, survival, and astroglial differentiation in vitro and in vivo. J Neurosci. 2005; 25: 5584-94.

18. Bodempudi V, Yamoutpoor F, Pan W, Dudek A Z, Esfandyari T, Piedra M, Babovick-Vuksanovic D, Woo R A, Mautner V F, Kluwe L, Clapp D W, De Vrie G H, Thomas S L, et al. Ral overactivation in malignant peripheral nerve sheath tumors. Mol Cell Biol. 2009; 29: 3964-74.
19. Reuss D E, Mucha J, Hagenlocher C, Ehemann V, Kluwe L, Mautner V, von Deimling A. Sensitivity of malignant peripheral nerve sheath tumor cells to TRAIL is augmented by loss of NF1 through modulation of MYC/MAD and is potentiated by curcumin through induction of ROS. PLoS One. 2013; 8: e57152.
20. Sun D, Tainsky M A, Haddad R. Oncogene Mutation Survey in MPNST Cell Lines Enhances the Dominant Role of Hyperactive Ras in NF1 Associated Pro-Survival and Malignancy. Transl Oncogenomics. 2012; 5: 1-7.
21. Gao G P, Alvira M R, Wang L, Calcedo R, Johnston J, Wilson J M. Novel adenoassociated viruses from rhesus monkeys as vectors for human gene therapy. Proc Natl Acad Sci U S A. 2002; 99: 11854-9.
22. Cearley C N, Wolfe J H. Transduction characteristics of adeno-associated virus vectors expressing cap serotypes 7, 8, 9, and Rh10 in the mouse brain. Mol Ther. 2006; 13: 528-37.
23. Cearley C N, Vandenberghe L H, Parente M K, Carnish E R, Wilson J M, Wolfe J H. Expanded repertoire of AAV vector serotypes mediate unique patterns of transduction in mouse brain. Mol Ther. 2008; 16: 1710-8.
24. Mays L E, Wilson J M. Identification of the murine AAVrh32.33 capsid-specific CD8+ T cell epitopes. J Gene Med. 2009; 11: 1095-102. 10.1002/jgm.1402.
25. Grimm D, Lee J S, Wang L, Desai T, Akache B, Storm T A, Kay M A. In vitro and in vivo gene therapy vector evolution via multispecies interbreeding and retargeting of adeno-associated viruses. J Virol. 2008; 82: 5887-911.
26. Wang Z, Ma H I, Li J, Sun L, Zhang J, Xiao X. Rapid and highly efficient transduction by double-stranded adeno-associated virus vectors in vitro and in vivo. Gene Ther. 2003; 10: 2105-11.
27. Zhu Y, Ghosh P, Charnay P, Burns D K, Parada L F. Neurofibromas in NF1: Schwann cell origin and role of tumor environment. Science. 2002; 296: 920-2.
28. Hancock J F, Cadwallader K, Paterson H, Marshall C J. A CAAX or a CAAL motif and a second signal are sufficient for plasma membrane targeting of ras proteins. EMBO J. 1991; 10: 4033-9.
29. Simanshu D K, Nissley D V, McCormick F. RAS Proteins and Their Regulators in Human Disease. Cell. 2017; 170: 17-33. 10.1016/j.cell.2017.06.009.
30. Weinmann J, Grimm D. Next-generation AAV vectors for clinical use: an ever accelerating race. Virus Genes. 2017; 53: 707-13.
31. Mao Y, Wang X, Yan R, Hu W, Li A, Wang S, Li H. Single point mutation in adenoassociated viral vectors-DJ capsid leads to improvement for gene delivery in vivo. BMC Biotechnol. 2016; 16: 1.
32. Hoyng S A, De Winter F, Gnavi S, van Egmond L, Attwell C L, Tannemaat M R, Verhaagen J, Malessy M J. Gene delivery to rat and human Schwann cells and nerve segments: a comparison of AAV 1-9 and lentiviral vectors. Gene Ther. 2015; 22: 767-80.
33. Scheffzek K, Shivalingaiah G. Ras-Specific GTPase-Activating Proteins-Structures, Mechanisms, and Interactions. Cold Spring Harb Perspect Med. 2018.
34. Ahmadian M R, Wiesmuller L, Lautwein A, Bischoff F R, Wittinghofer A. Structural differences in the minimal catalytic domains of the GTPase-activating proteins p120GAP and neurofibromin. J Biol Chem. 1996; 271: 16409-15.
35. Scheffzek K, Ahmadian M R, Wiesmuller L, Kabsch W, Stege P, Schmitz F, Wittinghofer A. Structural analysis of the GAP-related domain from neurofibromin and its implications. EMBO J. 1998; 17: 4313-27.
36. Stowe I B, Mercado E L, Stowe T R, Bell E L, Oses-Prieto J A, Hernandez H, Burlingame A L, McCormick F. A shared molecular mechanism underlies the human rasopathies Legius syndrome and Neurofibromatosis-1. Genes Dev. 2012; 26: 1421-6.
37. Hirata Y, Brems H, Suzuki M, Kanamori M, Okada M, Morita R, Llano-Rivas I, Ose T, Messiaen L, Legius E, Yoshimura A. Interaction between a Domain of the Negative Regulator of the Ras-ERK Pathway, SPRED1 Protein, and the GTPase-activating Protein-related Domain of Neurofibromin Is Implicated in Legius Syndrome and Neurofibromatosis Type 1. J Biol Chem. 2016; 291: 3124-34.
38. Dunzendorfer-Matt T, Mercado E L, Maly K, McCormick F, Scheffzek K. The neurofibromin recruitment factor Spred1 binds to the GAP related domain without affecting Ras inactivation. Proc Natl Acad Sci U S A. 2016; 113: 7497-502.
39. Morcos P, Thapar N, Tusneem N, Stacey D, Tamanoi F. Identification of neurofibromin mutants that exhibit allele specificity or increased Ras affinity resulting in suppression of activated ras alleles. Mol Cell Biol. 1996; 16: 2496-503.
40. Bai R Y, Staedtke V, Aprhys C M, Gallia G L, Riggins G J. Antiparasitic mebendazole shows survival benefit in 2 preclinical models of glioblastoma multiforme. Neuro Oncol. 2011; 13: 974-82.
41. Bai R Y, Dieter P, Peschel C, Morris S W, Duyster J. Nucleophosmin-anaplastic lymphoma kinase of large-cell anaplastic lymphoma is a constitutively active tyrosine kinase that utilizes phospholipase C-gamma to mediate its mitogenicity. Mol Cell Biol. 1998; 18(12)6951-61.
42. Li, H., Chang, L. J., Neubauer, D. R., Muir, D. F. & Wallace, M. R. Immortalization of human normal and NF1 neurofibroma Schwann cells. Lab Invest 96, 1105-1115 (2016).

### 43. Cox, A. D., Der C. J. & Philips M. R. Targeting RAS Membrane Association: Back to the Future for Anti-RAS Drug Discovery? Clin Cancer Res. 2015; 21(8):1819-27.

44. Wennerberg, K., Rossman, K. L. & Der, C. J. The Ras superfamily at a glance. J Cell Sci. 2005;118(Pt 5): 843-6.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A recombinant polypeptide comprising a neurofibromin 1 GTPase-activating protein-related domain (NF1-GRD) and a membrane-targeting amino acid sequence.

2. The recombinant polypeptide of claim 1 wherein said membrane-targeting amino acid sequence comprises a CAAX motif-containing partial or complete Ras HVR sequence.

3. The recombinant polypeptide of claim 1 wherein said NF1-GRD amino acid sequence comprises an amino acid sequence provided by SEQ ID NO: 2.

4. The recombinant polypeptide of claim 1 wherein said NF1-GRD amino acid sequence comprises an amino acid sequence from approximately amino acid 1150 to approximately amino acid 1600 of the amino acid sequence provided by SEQ ID NO: 2.

5. The recombinant polypeptide of claim 1 wherein said membrane-targeting amino acid sequence comprises the amino acid sequence GCMSCKCVLS (SEQ ID NO: 3).

6. The recombinant polypeptide of claim 1 wherein said membrane-targeting amino acid sequence is joined in-frame to the C-terminus of said NF1-GRD.

7. A recombinant nucleic acid comprising a nucleotide sequence encoding a NF1-GRD and a membrane-targeting amino acid sequence.

8. The recombinant nucleic acid of claim 7 wherein said nucleotide sequence encoding said NF1-GRD comprises a nucleotide sequence provided by SEQ ID NO: 1.

9. The recombinant nucleic acid of claim 7 wherein said nucleotide sequence encoding said NF1-GRD comprises a nucleotide sequence from approximately nucleotide 3448 to approximately nucleotide 4650 of the nucleotide sequence provided by SEQ ID NO: 1.

10. The recombinant nucleic acid of claim 7 wherein said nucleic acid comprises a nucleotide sequence encoding a CAAX motif-containing partial or complete Ras HVR.

11. The recombinant nucleic acid of claim 7 wherein said nucleic acid comprises a nucleotide sequence encoding the amino acid sequence GCMSCKCVLS (SEQ ID NO: 3).

12. The recombinant nucleic acid of claim 7 wherein said nucleotide sequence encodes said membrane-targeting amino acid sequence joined in-frame to the C-terminus of said NF1-GRD.

13. A vector comprising a nucleic acid comprising a recombinant nucleotide sequence encoding a NF1-GRD and a membrane-targeting amino acid sequence.

14. The vector of claim 13 wherein said vector is a gene-delivery vector.

15. The vector of claim 13 wherein said vector is an adeno-associated virus (AAV) vector.

16. The vector of claim 13 wherein said vector is an AAV1, AAV2, AAV3B, AAV6, or AAV-DJ AAV vector.

17. A cell comprising a recombinant nucleic acid comprising a nucleotide sequence encoding a NF1-GRD and a membrane-targeting amino acid sequence.

18. The cell of claim 17 wherein said recombinant nucleic acid is integrated into the genome.

19. The cell of claim 17 wherein said recombinant nucleic acid is present in a vector.

20. A tissue or organism comprising a cell of claim 17.

21. A virus comprising a recombinant nucleic acid comprising a nucleotide sequence encoding a NF1-GRD and a membrane-targeting amino acid sequence.

22. A cell expressing a recombinant nucleic acid comprising a nucleotide sequence encoding a NF1-GRD and a membrane-targeting amino acid sequence.

23. A cell expressing a recombinant polypeptide comprising a NF1-GRD and a membrane-targeting amino acid sequence.

24. The cell of claim 22 wherein said cell comprises aberrant Ras activity.

25. The cell of claim 23 wherein said cell comprises aberrant Ras activity.

26. The cell of claim 22 wherein said cell comprises a mutation in a NF1 gene.

27. The cell of claim 23 wherein said cell comprises a mutation in a NF1 gene.

28. The cell of claim 22 wherein said cell is a cancer or tumor cell.

29. The cell of claim 22 wherein said cell is a Schwann cell.

30. The cell of claim 23 wherein said cell is a Schwann cell.

31. The cell of claim 22 wherein said cell is a malignant peripheral nerve sheath tumor (MPNST) cell.

32. The cell of claim 23 wherein said cell is a MPNST cell.

33. A subject having a Rasopathy comprising the cell of claim 22.

34. A subject having a Rasopathy comprising the cell of claim 23.

35. A subject having neurofibromatosis 1 comprising the cell of claim 22.

36. A subject having neurofibromatosis 1 comprising the cell of claim 23.

37. A subject having a MPNST comprising the cell of claim 22.

38. A subject having a MPNST comprising the cell of claim 23.

39. A pharmaceutical composition comprising a recombinant nucleic acid comprising a nucleotide sequence encoding a NF1-GRD and a membrane-targeting amino acid sequence.

40. The pharmaceutical composition of claim 39 wherein a gene-delivery vector comprises said recombinant nucleic acid.

41. The pharmaceutical composition of claim 39 wherein said recombinant nucleic acid is provided in a therapeutically effective dose.

42. The pharmaceutical composition of claim 39 formulated for administration to a subject or to a tissue.

43. The pharmaceutical composition of claim 39 formulated in a gene-delivery virus.

44. A kit comprising a pharmaceutical composition comprising a recombinant nucleic acid comprising a nucleotide sequence encoding a NF1-GRD and a membrane-targeting amino acid sequence and a means for administration.

45. A method of treating a subject having a Rasopathy, the method comprising administering a recombinant nucleic acid comprising a nucleotide sequence encoding a NF1-GRD and a membrane-targeting amino acid sequence to a subject.

46. The method of claim 45 wherein said administering reduces Ras activity in the subject.

47. The method of claim 45, wherein the subject has or is at risk of developing a neurofibromatosis or a MPNST.

48. The method of claim 45, wherein the subject has a mutation in the NF1 gene.

49. The method of claim 45, wherein said administration results in a reduced tumor load in the subject.

50. The method of claim 45, wherein the subject is a human.

51. The method of claim 45, wherein the administering comprises administering said recombinant nucleic acid in combination with an additional therapeutic agent or medical intervention.

52. The method of claim 45, further comprising testing the subject for a NF1 mutation.

53. The method of claim 45, further comprising testing the subject for aberrant Ras activity.

54. The method of claim 45, further comprising testing the subject for a cancer.

55. The method of claim 45, further comprising testing the subject for a neurofibroma or MPNST.

56. The method of claim 52, further comprising the step of administering a second dose of said recombinant nucleic acid after the testing step.

57. The method of claim 53, further comprising the step of administering a second dose of said recombinant nucleic acid after the testing step.

58. The method of claim 54, further comprising the step of administering a second dose of said recombinant nucleic acid after the testing step.

59. The method of claim 55, further comprising the step of administering a second dose of said recombinant nucleic acid after the testing step.

60. Use of a recombinant nucleic acid comprising a nucleotide sequence encoding a NF1-GRD and a membrane-targeting amino acid sequence to prepare a medicament.

61. Use of a recombinant nucleic acid comprising a nucleotide sequence encoding a NF1-GRD and a membrane-targeting amino acid sequence to prepare a medicament for treating a subject having a Rasopathy, neurofibromatosis type 1, and/or a MPNST disease.

62. Use of a recombinant nucleic acid comprising a nucleotide sequence encoding a NF1-GRD and a membrane-targeting amino acid sequence to provide a transgenic organism.

63. Use of a recombinant nucleic acid comprising a nucleotide sequence encoding a NF1-GRD and a membrane-targeting amino acid sequence to provide a gene therapy construct.

64. Use of a recombinant nucleic acid comprising a nucleotide sequence encoding a NF1-GRD and a membrane-targeting amino acid sequence in research.

65. Use of a recombinant nucleic acid comprising a nucleotide sequence encoding a NF1-GRD and a membrane-targeting amino acid sequence in studying disease in a model system (e.g., a mammal (e.g., a mouse, rat, dog) and/or a cell culture system).

66. The use of claim 61 wherein said medicament comprises a virus.


SEQUENCE LISTING (64 sequences)

SEQ ID NO: 1 - DNA, 12381 bp, Homo sapiens
aatctctagctcgctcgcgctccctctccccgggccgtggaaaggatcccacttccggtggggtgtcatggcggcgtctcggactgtgatggctgtggggagacggcgctagtggggagagcgaccaagaggccccctcccctccccgggtccccttcccctatccccctccccccagcctccttgccaacgccccctttccctctccccctcccgctcggcgctgaccccccatccccacccccgtgggaacactgggagcctgcactccacagaccctctccttgcctcttccctcacctcagcctccgctccccgccctcttcccggcccagggcgccggcccacccttccctccgccgccccccggccgcggggaggacatggccgcgcacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgtttataccaggtggttagccagcgtttccctcagaacagcatcggtgcagtaggaagtgccatgttcctcagatttatcaatcctgccattgtctcaccgtatgaagcagggattttagataaaaagccaccacctagaatcgaaaggggcttgaagttaatgtcaaagatacttcagagtattgccaatcatgttctcttcacaaaagaagaacatatgcggcctttcaatgattttgtgaaaagcaactttgatgcagcacgcaggtttttccttgatatagcatctgattgtcctacaagtgatgcagtaaatcatagtctttccttcataagtgacggcaatgtgcttgctttacatcgtctactctggaacaatcaggagaaaattgggcagtatctttccagcaacagggatcataaagctgttggaagacgaccttttgataagatggcaacacttcttgcatacctgggtcctccagagcacaaacctgtggcagatacacactggtccagccttaaccttaccagttcaaagtttgaggaatttatgactaggcatcaggtacatgaaaaagaagaattcaaggctttgaaaacgttaagtattttctaccaagctgggacttccaaagctgggaatcctattttttattatgttgcacggaggttcaaaactggtcaaatcaatggtgatttgctgatataccatgtcttactgactttaaagccatattatgcaaagccatatgaaattgtagtggaccttacccataccgggcctagcaatcgctttaaaacagactttctctctaagtggtttgttgtttttcctggctttgcttacgacaacgtctccgcagtctatatctataactgtaactcctgggtcagggagtacaccaagtatcatgagcggctgctgactggcctcaaaggtagcaaaaggcttgttttcatagactgtcctgggaaactggctgagcacatagagcatgaacaacagaaactacctgctgccaccttggctttagaagaggacctgaaggtattccacaatgctctcaagctagctcacaaagacaccaaagtttctattaaagttggttctactgctgtccaagtaacttcagcagagcgaacaaaagtcctagggcaatcagtctttctaaatgacatttattatgcttcggaaattgaagaaatctgcctagtagatgagaaccagttcaccttaaccattgcaaaccagggcacgccgctcaccttcatgcaccaggagtgtgaagccattgtccagtctatcattcatatccggacccgctgggaactgtcacagcccgactctatcccccaacacaccaagattcggccaaaagatgtccctgggacactgctcaatatcgcattacttaatttaggcagttctgacccgagtttacggtcagctgcctataatcttctgtgtgccttaacttgtacctttaatttaaaaatcgagggccagttactagagacatcaggtttatgtatccctgccaacaacaccctctttattgtctctattagtaagacactggcagccaatgagccacacctcacgttagaatttttggaagagtgtatttctggatttagcaaatctagtattgaattgaaacacctttgtttggaatacatgactccatggctgtcaaatctagttcgtttttgcaagcataatgatgatgccaaacgacaaagagttactgctattcttgacaagctgataacaatgaccatcaatgaaaaacagatgtacccatctattcaagcaaaaatatggggaagccttgggcagattacagatctgcttgatgttgtactagacagtttcatcaaaaccagtgcaacaggtggcttgggatcaataaaagctgaggtgatggcagatactgctgtagctttggcttctggaaatgtgaaattggtttcaagcaaggttattggaaggatgtgcaaaataattgacaagacatgcttatctccaactcctactttagaacaacatcttatgtgggatgatattgctattttagcacgctacatgctgatgctgtccttcaacaattcccttgatgtggcagctcatcttccctacctcttccacgttgttactttcttagtagccacaggtccgctctcccttagagcttccacacatggactggtcattaatatcattcactctctgtgtacttgttcacagcttcattttagtgaagagaccaagcaagttttgagactcagtctgacagagttctcattacccaaattttacttgctgtttggcattagcaaagtcaagtcagctgctgtcattgccttccgttccagttaccgggacaggtcattctctcctggctcctatgagagagagacttttgctttgacatccttggaaacagtcacagaagctttgttggagatcatggaggcatgcatgagagatattccaacgtgcaagtggctggaccagtggacagaactagctcaaagatttgcattccaatataatccatccctgcaaccaagagctcttgttgtctttgggtgtattagcaaacgagtgtctcatgggcagataaagcagataatccgtattcttagcaaggcacttgagagttgcttaaaaggacctgacacttacaacagtcaagttctgatagaagctacagtaatagcactaaccaaattacagccacttcttaataaggactcgcctctgcacaaagccctcttttgggtagctgtggctgtgctgcagcttgatgaggtcaacttgtattcagcaggtaccgcacttcttgaacaaaacctgcatactttagatagtctccgtatattcaatgacaagagtccagaggaagtatttatggcaatccggaatcctctggagtggcactgcaagcaaatggatcattttgttggactcaatttcaactctaactttaactttgcattggttggacaccttttaaaagggtacaggcatccttcacctgctattgttgcaagaacagtcagaattttacatacactactaactctggttaacaaacacagaaattgtgacaaatttgaagtgaatacacagagcgtggcctacttagcagctttacttacagtgtctgaagaagttcgaagtcgctgcagcctaaaacatagaaagtcacttcttcttactgatatttcaatggaaaatgttcctatggatacatatcccattcatcatggtgacccttcctataggacactaaaggagactcagccatggtcctctcccaaaggttctgaaggataccttgcagccacctatccaactgtcggccagaccagtccccgagccaggaaatccatgagcctggacatggggcaaccttctcaggccaacactaagaagttgcttggaacaaggaaaagttttgatcacttgatatcagacacaaaggctcctaaaaggcaagaaatggaatcagggatcacaacaccccccaaaatgaggagagtagcagaaactgattatgaaatggaaactcagaggatttcctcatcacaacagcacccacatttacgtaaagtttcagtgtctgaatcaaatgttctcttggatgaagaagtacttactgatccgaagatccaggcgctgcttcttactgttctagctacactggtaaaatataccacagatgagtttgatcaacgaattctttatgaatacttagcagaggccagtgttgtgtttcccaaagtctttcctgttgtgcataatttgttggactctaagatcaacaccctgttatcattgtgccaagatccaaatttgttaaatccaatccatggaattgtgcagagtgtggtgtaccatgaagaatccccaccacaataccaaacatcttacctgcaaagttttggttttaatggcttgtggcggtttgcaggaccgttttcaaagcaaacacaaattccagactatgctgagcttattgttaagtttcttgatgccttgattgacacgtacctgcctggaattgatgaagaaaccagtgaagaatccctcctgactcccacatctccttaccctcctgcactgcagagccagcttagtatcactgccaaccttaacctttctaattccatgacctcacttgcaacttcccagcattccccaggaatcgacaaggagaacgttgaactctcccctaccactggccactgtaacagtggacgaactcgccacggatccgcaagccaagtgcagaagcaaagaagcgctggcagtttcaaacgtaatagcattaagaagatcgtgtgaagcttgcttgctttcttttttaaaatcaacttaacatgggctcttcactagtgaccccttccctgtccttgccctttccccccatgttgtaatgctgcacttcctgttttataatgaacccatccggtttgccatgttgccagatgatcaactcttcgaagccttgcctaaatttaatgctgccttttctttaactttttttcttctacttttggcgtgtatctggtatatgtaagtgttcagaacaactgcaaagaaagtgggaggtcaggaaacttttaactgagaaatctcaattgtaagagaggatgaattcttgaatactgctactactggccagtgatgaaagccatttgcacagagctctgccttctgtggttttcccttcttcatcctacagagtaaagtgttagtcctatttatacatttttcaagatacaagtttatgagagaaatagtattataaccccagtatgtttaatcttttagctgtggactttttttttaaccgtacaaaactgaaagaaccatagaggtcaagcctcagtgacttgacaccataaagccacagacaaggtacttgggggggagggcagggaaatttcatattttatagtggattcttaagaaatactaacacttgagtattagcaataattacaggaaaataagtgcgaccacatatatcttaacattactgaattaaaactatggcttctaagtccttatccaaactcagtcatccaaactagtttatttttttctccagttgattatcttttaatttttaattttgctaaaggtggtttttttgtgttttgttttttgtaaaccaaaactatactaagtatagtaattatatatatatatatattttttcccctccccctcttctttcctaactaattctgagcagggtaatcagtgaacaaagtgttgaaaattgttcccagaaggtaattttcatagatgtttgcattagctccatagcaaaatggaatggtacgtgacatttagggtagctgatatttttattttgttaaataatttccaagaatagagtatggtgtatattataaatttctttgataagatgtattttgaatgtcttttaatcttcctcctcctctccaaaaaaatcagaaacctctttaagaaaacatgtaggttatatatgctagaattgcatttaatcactgtgaaaagactggtcagcctgcattagtatgacagtaggggggctgttagaattgctgctatactggtggtatggattatcatggcattggaattttcatagtaatgcagatccaatttctttgtggtacctgcagtttacaaaataatttgacttcagtgagcatattggtatctggatgttccaatttagaactaaaccatatttattacaaaaagatattaatccctctactcccaggttccctttatatgttaagatataatggctttgaggggggaaaaaataaacctaggggagaggggagtttcctgtagtgctgtttcattagaggatttcagtaaattaaattccacagctaattcaataaataatggtacatttaagtgttctgattttaataatatatttcacatttatccacacagtaacaatgtaatatgttaatgtaaataaaattggttttgatactcagaaataacaagaatttaattttttaaatttgtttacagtcctgggaaaagtaagaattatttgccaaaataagaggaaagaaaaccttagtattattaatgagtttaccatagaattgttggaaatactgaagacaggtgcaatttactaaacttttgtttttaaactattgtagaggctgcattagaagaaaatgtttataatgacagagcaactatgactatataaaaaagctgaaattagaactgtgtttagaaatagatcagtaacccagtgccaaggatgccaagctgccaccatggtcttggctctcccacaacccagtgtttctggggtaagtttcacagtttctaggccctggaatagcaggcagtgtaagcctttgataactttagttcgatgtttttcttgtttttgtttgttggtttggtgcatatgatagtgggtgttatgctattttgctcttcccatcaaaataaagaaacttccagaggtttactgttaaaaatactgatatttccataaacgggtttaccaagggtgtagtatttcataccgcctgaaatgatcagcattggcacaaatcaaaattcagccgcctttgaaatgcaaaaatacctttgactagtaagtacatcctaggagtttgaaaacttaactaaggtttaaaatttaccttgtttaaagaacttctgacttttgaggaaaatctagctttccaagtaactaaaatgtacatgagataaacctctcaccactatgtgtcccttgagaaatgcaacacttttttagtcttcatacttgtaatctataaaagaaattctgaagtttagaccaagttgcccatttctgcgtaattgacataagttctgttaaaaatattataagtaattcgtttcggtttgtagatgtttcccctgacttgttaaagaggaaaccaggaactcagtcatgtttttgtcctggataatctacctgttatgccagtactcccatccgaggggcatgcccttagttgcccagatggagatgcagttcagtagatttggggcaaagtggctacagctctgtcttccattcactcaacacctgttcatgactgagccaggtgcccaggacacatcctaaacagtcagcttctatcctgtgtcctagttggggagacagagtgccagccagcaaccctcccaggtttgtaggttttaggggttttcagttttgtttgggttttttgttttttgtttttgtttctacatccttccccgactcccaggcataatgaggcatgtcttactcaatgttatgcaatggatttaggcaaaaattcattcttagtgtcagccacacaattttttttaatgcagtatattcacctgtaaatagtttgtgtaaaatttgacaaaaaaagtatatttactatactgtaaatatatgtgatgatatattgtattattttgcttttttgtaaagcagttagttgctgcacatggataacaacaaaaatttgattattctcgtgttagtattgttaacttctttttgcgactgcgttacatcatttaaagaaaatgctgtgtattgtaaacttaaattgtatatgataacttactgtcctttccatccgggcctaaactttggcagttcctttgtctacaaccttgttaatactgtaaacagttgtacgccagcaggaaaaatactgcccaacagacaaaatcgatcattgtaggggaaaatcatagaaatccatttcagatctttattgttcctcaccccattttcctccttgtgtatgtacttcccccacccccctttttttaagtaaaatgtaaattcaatctgctctaagaaaaaaaaaaaaaaaaaaaa

SEQ ID NO: 2 - protein, 2818 aa, Homo sapiens
MAAHRPVEWVQAVVSRFDEQLPIKTGQQNTHTKVSTEHNKECLINISKYKFSLVISGLTTILKNVNNMRIFGEAAEKNLYLSQLIILDTLEKCLAGQPKDTMRLDETMLVKQLLPEICHFLHTCREGNQHAAELRNSASGVLFSLSCNNFNAVFSRISTRLQELTVCSEDNVDVHDIELLQYINVDCAKLKRLLKETAFKFKALKKVAQLAVINSLEKAFWNWVENYPDEFTKLYQIPQTDMAECAEKLFDLVDGFAESTKRKAAVWPLQIILLILCPEIIQDISKDVVDENNMNKKLFLDSLRKALAGHGGSRQLTESAAIACVKLCKASTYINWEDNSVIFLLVQSMVVDLKNLLFNPSKPFSRGSQPADVDLMIDCLVSCFRISPHNNQHFKICLAQNSPSTFHYVLVNSLHRIITNSALDWWPKIDAVYCHSVELRNMFGETLHKAVQGCGAHPAIRMAPSLTFKEKVTSLKFKEKPTDLETRSYKYLLLSMVKLIHADPKLLLCNPRKQGPETQGSTAELITGLVQLVPQSHMPEIAQEAMEALLVLHQLDSIDLWNPDAPVETFWEISSQMLFYICKKLTSHQMLSSTEILKWLREILICRNKFLLKNKQADRSSCHFLLFYGVGCDIPSSGNTSQMSMDHEELLRTPGASLRKGKGNSSMDSAAGCSGTPPICRQAQTKLEVALYMFLWNPDTEAVLVAMSCFRHLCEEADIRCGVDEVSVHNLLPNYNTFMEFASVSNMMSTGRAALQKRVMALLRRIEHPTAGNTEAWEDTHAKWEQATKLILNYPKAKMEDGQAAESLHKTIVKRRMSHVSGGGSIDLSDTDSLQEWINMTGFLCALGGVCLQQRSNSGLATYSPPMGPVSERKGSMISVMSSEGNADTPVSKFMDRLLSLMVCNHEKVGLQIRTNVKDLVGLELSPALYPMLFNKLKNTISKFFDSQGQVLLTDTNTQFVEQTIAIMKNLLDNHTEGSSEHLGQASIETMMLNLVRYVRVLGNMVHAIQIKTKLCQLVEVMMARRDDLSFCQEMKFRNKMVEYLTDWVMGTSNQAADDDVKCLTRDLDQASMEAVVSLLAGLPLQPEEGDGVELMEAKSQLFLKYFTLFMNLLNDCSEVEDESAQTGGRKRGMSRRLASLRHCTVLAMSNLLNANVDSGLMHSIGLGYHKDLQTRATFMEVLTKILQQGTEFDTLAETVLADRFERLVELVTMMGDQGELPIAMALANVVPCSQWDELARVLVTLFDSRHLLYQLLWNMFSKEVELADSMQTLFRGNSLASKIMTFCFKVYGATYLQKLLDPLLRIVITSSDWQHVSFEVDPTRLEPSESLEENQRNLLQMTEKFFHAIISSSSEFPPQLRSVCHCLYQVVSQRFPQNSIGAVGSAMFLRFINPAIVSPYEAGILDKKPPPRIERGLKLMSKILQSIANHVLFTKEEHMRPFNDFVKSNFDAARRFFLDIASDCPTSDAVNHSLSFISDGNVLALHRLLWNNQEKIGQYLSSNRDHKAVGRRPFDKMATLLAYLGPPEHKPVADTHWSSLNLTSSKFEEFMTRHQVHEKEEFKALKTLSIFYQAGTSKAGNPIFYYVARRFKTGQINGDLLIYHVLLTLKPYYAKPYEIVVDLTHTGPSNRFKTDFLSKWFVVFPGFAYDNVSAVYIYNCNSWVREYTKYHERLLTGLKGSKRLVFIDCPGKLAEHIEHEQQKLPAATLALEEDLKVFHNALKLAHKDTKVSIKVGSTAVQVTSAERTKVLGQSVFLNDIYYASEIEEICLVDENQFTLTIANQGTPLTFMHQECEAIVQSIIHIRTRWELSQPDSIPQHTKIRPKDVPGTLLNIALLNLGSSDPSLRSAAYNLLCALTCTFNLKIEGQLLETSGLCIPANNTLFIVSISKTLAANEPHLTLEFLEECISGFSKSSIELKHLCLEYMTPWLSNLVRFCKHNDDAKRQRVTAILDKLITMTINEKQMYPSIQAKIWGSLGQITDLLDVVLDSFIKTSATGGLGSIKAEVMADTAVALASGNVKLVSSKVIGRMCKIIDKTCLSPTPTLEQHLMWDDIAILARYMLMLSFNNSLDVAAHLPYLFHVVTFLVATGPLSLRASTHGLVINIIHSLCTCSQLHFSEETKQVLRLSLTEFSLPKFYLLFGISKVKSAAVIAFRSSYRDRSFSPGSYERETFALTSLETVTEALLEIMEACMRDIPTCKWLDQWTELAQRFAFQYNPSLQPRALVVFGCISKRVSHGQIKQIIRILSKALESCLKGPDTYNSQVLIEATVIALTKLQPLLNKDSPLHKALFWVAVAVLQLDEVNLYSAGTALLEQNLHTLDSLRIFNDKSPEEVFMAIRNPLEWHCKQMDHFVGLNFNSNFNFALVGHLLKGYRHPSPAIVARTVRILHTLLTLVNKHRNCDKFEVNTQSVAYLAALLTVSEEVRSRCSLKHRKSLLLTDISMENVPMDTYPIHHGDPSYRTLKETQPWSSPKGSEGYLAATYPTVGQTSPRARKSMSLDMGQPSQANTKKLLGTRKSFDHLISDTKAPKRQEMESGITTPPKMRRVAETDYEMETQRISSSQQHPHLRKVSVSESNVLLDEEVLTDPKIQALLLTVLATLVKYTTDEFDQRILYEYLAEASVVFPKVFPVVHNLLDSKINTLLSLCQDPNLLNPIHGIVQSVVYHEESPPQYQTSYLQSFGFNGLWRFAGPFSKQTQIPDYAELIVKFLDALIDTYLPGIDEETSEESLLTPTSPYPPALQSQLSITANLNLSNSMTSLATSQHSPGIDKENVELSPTTGHCNSGRTRHGSASQVQKQRSAGSFKRNSIKKIV

SEQ ID NO: 3 - protein, 10 aa, Artificial Sequence
  Synthetic
GCMSCKCVLS

SEQ ID NO: 4 - DNA, 27 bp, Artificial sequence
  Synthetic
tacccatacgatgttccagattacgct

SEQ ID NO: 5 - DNA, 27 bp, Artificial sequence
  Synthetic
tatccatatgatgttccagattatgct

SEQ ID NO: 6 - protein, 9 aa, Artificial Sequence
  Synthetic
YPYDVPDYA

SEQ ID NO: 7 - protein, 18 aa, Artificial Sequence
  Synthetic
YPYDVPDYAYPYDVPDYA

SEQ ID NO: 8 - protein, 27 aa, Artificial Sequence
  Synthetic
YPYDVPDYAYPYDVPDYAYPYDVPDYA

SEQ ID NO: 9 - protein, 36 aa, Artificial Sequence
  Synthetic
YPYDVPDYAYPYDVPDYAYPYDVPDYAYPYDVPDYA

SEQ ID NO: 10 - protein, 24 aa, Homo sapiens
HKLRKLNPPDESGPGCMSCKCVLS

SEQ ID NO: 11 - protein, 24 aa, Homo sapiens
YRMKKLNSSDDGTQGCMGLPCVVM

SEQ ID NO: 12 - protein, 24 aa, Homo sapiens
YRLKKISKEEKTPGCVKIKKCIIM

SEQ ID NO: 13 - protein, 24 aa, Homo sapiens
KHKEKMSKDGKKKKKKSKTKCVIM

SEQ ID NO: 14 - protein, 10 aa, Homo sapiens
GCMSCKCVLS

SEQ ID NO: 15 - protein, 21 aa, Homo sapiens
EKMSKDGKKKKKKSKTKCVIM

SEQ ID NO: 16 - protein, 22 aa, Homo sapiens
LKKISKEEKTPGCVKIKKCIIM

SEQ ID NO: 17 - protein, 24 aa, Homo sapiens
HKLRKLNPPDESGPGCMSCKCVLS

SEQ ID NO: 18 - protein, 24 aa, Homo sapiens
YRMKKLNSSDDGTQGCMGLPCVVM

SEQ ID NO: 19 - protein, 24 aa, Homo sapiens
YRLKKISKEEKTPGCVKIKKCIIM

SEQ ID NO: 20 - protein, 24 aa, Homo sapiens
KHKEKMSKDGKKKKKKSKTKCVIM

SEQ ID NO: 21 - protein, 25 aa, Homo sapiens
QHKLRKLNPPDESGPGCMSCKCVLS

SEQ ID NO: 22 - protein, 25 aa, Homo sapiens
QYRMKKLNSSDDGTQGCMGLPCVVM

SEQ ID NO: 23 - protein, 25 aa, Homo sapiens
QYRLKKISKEEKTPGCVKIKKCIIM

SEQ ID NO: 24 - protein, 24 aa, Homo sapiens
KHKEKMSKDGKKKKKKSKTKCVIM

SEQ ID NO: 25 - protein, 24 aa, Artificial sequence
  Synthetic
QHKLRKLNPPDESGPGCMSCKCVS

SEQ ID NO: 26 - protein, 25 aa, Homo sapiens
QHKLRKLNPPDESGPGCMSCKCVLS

SEQ ID NO: 27 - protein, 25 aa, Chlorocebus sabaeus
QHKLRKLNPPDESGPGCMSCKCVLS

SEQ ID NO: 28 - protein, 25 aa, Mus musculus
QHKLRKLNPPDESGPGCMSCKCVLS

SEQ ID NO: 29 - protein, 25 aa, Rattus norvegicus
QHKLRKLNPPDESGPGCMSCKCVLS

SEQ ID NO: 30 - protein, 24 aa, Gallus gallus
QHKLRLNPPDESGPGCMNCKCVIS

SEQ ID NO: 31 - protein, 24 aa, Alligator mississippiensis
QHKLRLNPPDESGPGCMNCKCVIS

SEQ ID NO: 32 - protein, 24 aa, Chelonia mydas
QHKLRLNPPDESGPGCMNCKCVIS

SEQ ID NO: 33 - protein, 25 aa, Xenopus tropicalis
QHKLRKLNPPDESEKGCLNCKCVVS

SEQ ID NO: 34 - protein, 25 aa, Danio rerio
QHKMRKLNPPDESGQDCMSCRCVVS

SEQ ID NO: 35 - protein, 23 aa, Artificial sequence
  Synthetic
QYRMKKLNSSEDGQGCMGLPCVM

SEQ ID NO: 36 - protein, 25 aa, Homo sapiens
QYRMKKLNSSDDGTQGCMGLPCVVM

SEQ ID NO: 37 - protein, 25 aa, Chlorocebus sabaeus
QYRMKKLNSSDDGTQGCMGLPCVVM

SEQ ID NO: 38 - protein, 25 aa, Mus musculus
QYRMKKLNSSDDGTQGCMGLPCVLM

SEQ ID NO: 39 - protein, 25 aa, Rattus norvegicus
QYRMKKLNSSEDGTQGCMGLPCVVM

SEQ ID NO: 40 - protein, 25 aa, Gallus gallus
QYRMKKLNSNEDGNQGCMGLSCIVM

SEQ ID NO: 41 - protein, 25 aa, Alligator mississippiensis
QYRMKKLNSSEDGNQGCMGVSCIMM

SEQ ID NO: 42 - protein, 25 aa, Chelonia mydas
QYRMKKLNSSEDGNQGCMGLSCIVM

SEQ ID NO: 43 - protein, 25 aa, Xenopus tropicalis
QYRMKKLDSSEDNNQGCIRIPCKLM

SEQ ID NO: 44 - protein, 24 aa, Danio rerio
YRMKKLNSREDRKQGCLGVSCEVM

SEQ ID NO: 45 - protein, 23 aa, Artificial sequence
  Synthetic
QYRLKKISKEEKTPGCVKIKKCM

SEQ ID NO: 46 - protein, 25 aa, Homo sapiens
QYRLKKISKEEKTPGCVKIKKCIIM

SEQ ID NO: 47 - protein, 25 aa, Chlorocebus sabaeus
QYRLKKISKEEKTPGCVKIKKCIIM

SEQ ID NO: 48 - protein, 25 aa, Mus musculus
QYRLKKISKEEKTPGCVKIKKCVIM

SEQ ID NO: 49 - protein, 25 aa, Rattus norvegicus
QYRLKKISKEEKTPGCVKIKKCVIM

SEQ ID NO: 50 - protein, 25 aa, Gallus gallus
QYRVKKISKEEKTPGCMKIKKCLVM

SEQ ID NO: 51 - protein, 25 aa, Alligator mississippiensis
QYRVKKISKEEKTPGCVKIKKCLVM

SEQ ID NO: 52 - protein, 25 aa, Chelonia mydas
QYRVKKISKEEKTPGCVKIKKCLVM

SEQ ID NO: 53 - protein, 25 aa, Xenopus tropicalis
QFRLTKMSKEEKTPGCVKFKKCRVM

SEQ ID NO: 54 - protein, 25 aa, Danio rerio
EYRLRKLSKEEKTTQCIKLKKCVLM

SEQ ID NO: 55 - protein, 23 aa, Artificial sequence
  Synthetic
KHKEKMSKDGKKKKKKSKTKCIM

SEQ ID NO: 56 - protein, 24 aa, Homo sapiens
KHKEKMSKDGKKKKKKSKTKCVIM

SEQ ID NO: 57 - protein, 24 aa, Chlorocebus sabaeus
KHKEKMSKDGKKKKKKSKTKCVIM

SEQ ID NO: 58 - protein, 24 aa, Mus musculus
KHKEKMSKDGKKKKKKSRTRCTVM

SEQ ID NO: 59 - protein, 24 aa, Rattus norvegicus
KHKEKMSKDGKKKKKKSRTRCTVM

SEQ ID NO: 60 - protein, 24 aa, Gallus gallus
KHKEKMSKDGKKKKKKTKTKCIIM

SEQ ID NO: 61 - protein, 24 aa, Alligator mississippiensis
KHKEKMSKDGKKKKKKTKTKCIIM

SEQ ID NO: 62 - protein, 24 aa, Chelonia mydas
KHKEKMSKDGKKKKKKTKTKCIIM

SEQ ID NO: 63 - protein, 24 aa, Xenopus tropicalis
KHKEKMSKEGKKKKKKSKTKCALM

SEQ ID NO: 64 - protein, 23 aa, Danio rerio
KHKEKMSKGDKKKKKSKTKCSIL